SEQUENCE LISTING (11 sequences)

SEQ ID NO: 1 - protein, 479 aa, Escherichia coli
MSVPVQHPMYIDGQFVTWRGDAWIDVVNPATEAVISRIPDGQAEDARKAIDAAERAQPEWEALPAIERASWLRKISAGIRERASEISALIVEEGGKIQQLAEVEVAFTADYIDYMAEWARRYEGEIIQSDRPGENILLFKRALGVTTGILPWNFPFFLIARKMAPALLTGNTIVIKPSEFTPNNAIAFAKIVDEIGLPRGVFNLVLGRGETVGQELAGNPKVAMVSMTGSVSAGEKIMATAAKNITKVCLELGGKAPAIVMDDADLELAVKAIVDSRVINSGQVCNCAERIYVQKGIYDQFVNRLGEAMQAVQFGNPAERNDIAMGPLINAAALERVEQKVARAVEEGARVALGGKAVEGKGYYYPPTLLLDVLQEMSIMHEETFGPVLPVVAFDTLEEAISMANDSDYGLTSSIYTQNLNVAMKAIKGLKFGETYINRENFEAMQGFHAGWRKSGIGGADGKHGLHEYLQTQVVYLQS

SEQ ID NO: 2 - protein, 382 aa, Escherichia coli
MANRMILNETAWFGRGAVGALTDEVKRRGYQKALIVTDKTLVQCGVVAKVTDKMDAAGLAWAIYDGVVPNPTITVVKEGLGVFQNSGADYLIAIGGGSPQDTCKAIGIISNNPEFADVRSLEGLSPTNKPSVPILAIPTTAGTAAEVTINYVITDEEKRRKFVCVDPHDIPQVAFIDADMMDGMPPALKAATGVDALTHAIEGYITRGAWALTDALHIKAIEIIAGALRGSVAGDKDAGEEIALGQYVAGMGFSNVGLGLVHGMAHPLGAFYNTPHGVANAILLPHVMRYNADFTGEKYRDIARVMGVKVEGMSLEEARNAAVEAVFALNRDVGIPPHLRDVGVRKEDIPALAQAALNDVCTGGNPREATLEDIVELYHTAW

SEQ ID NO: 3 - protein, 462 aa, Escherichia coli
MTITPATHAISINPATGEQLSVLPWAGADDIENALQLAAAGFRDWRETNIDYRAEKLRDIGKALRARSEEMAQMITREMGKPINQARAEVAKSANLCDWYAEHGPAMLKAEPTLVENQQAVIEYRPLGTILAIMPWNFPLWQVMRGAVPIILAGNGYLLKHAPNVMGCAQLIAQVFKDAGIPQGVYGWLNADNDGVSQMIKDSRIAAVTVTGSVRAGAAIGAQAGAALKKCVLELGGSDPFIVLNDADLELAVKAAVAGRYQNTGQVCAAAKRFIIEEGIASAFTERFVAAAAALKMGDPRDEENALGPMARFDLRDELHHQVEKTLAQGARLLLGGEKMAGAGNYYPPTVLANVTPEMTAFREEMFGPVAAITIAKDAEHALELANDSEFGLSATIFTTDETQARQMAARLECGGVFINGYCASDARVAFGGVKKSGFGRELSHFGLHEFCNIQTVWKDRI

SEQ ID NO: 4 - protein, 495 aa, Escherichia coli
MNFHHLAYWQDKALSLAIENRLFINGEYTAAAENETFETVDPVTQAPLAKIARGKSVDIDRAMSAARGVFERGDWSLSSPAKRKAVLNKLADLMEAHAEELALLETLDTGKPIRHSLRDDIPGAARAIRWYAEAIDKVYGEVATTSSHELAMIVREPVGVIAAIVPWNFPLLLTCWKLGPALAAGNSVILKPSEKSPLSAIRLAGLAKEAGLPDGVLNVVTGFGHEAGQALSRHNDIDAIAFTGSTRTGKQLLKDAGDSNMKRVWLEAGGKSANIVFADCPDLQQAASATAAGIFYNQGQVCIAGTRLLLEESIADEFLALLKQQAQNWQPGHPLDPATTMGTLIDCAHADSVHSFIREGESKGQLLLDGRNAGLAAAIGPTIFVDVDPNASLSREEIFGPVLVVTRFTSEEQALQLANDSQYGLGAAVWTRDLSRAHRMSRRLKAGSVFVNNYNDGDMTVPFGGYKQSGNGRDKSLHALEKFTELKTIWISLEA

SEQ ID NO: 5 - protein, 490 aa, Escherichia coli
MSRMAEQQLYIHGGYTSATSGRTFETINPANGNVLATVQAAGREDVDRAVKSAQQGQKIWAAMTAMERSRILRRAVDILRERNDELAKLETLDTGKAYSETSTVDIVTGADVLEYYAGLIPALEGSQIPLRETSFVYTRREPLGVVAGIGAWNYPIQIALWKSAPALAAGNAMIFKPSEVTPLTALKLAEIYSEAGLPDGVFNVLPGVGAETGQYLTEHPGIAKVSFTGGVASGKKVMANSAASSLKEVTMELGGKSPLIVFDDADLDLAADIAMMANFFSSGQVCTNGTRVFVPAKCKAAFEQKILARVERIRAGDVFDPQTNFGPLVSFPHRDNVLRYIVKGKEEGARVLCGGDVLKGDDFDNGAWVAPTVFTDCSDDMTIVREEIFGPVMSILTYESEDEVIRRANDTDYGLAAGIVTADLNRAHRVIHQLEAGICWINTWGESPAEMPVGGYKHSGIGRENGVMTLQSYTQVKSIQVEMAKFQSIF

SEQ ID NO: 6 - protein, 467 aa, Escherichia coli
MNQQDIEQVVKAVLLKMQSSDTPPAAVHEMGVFASLDDAVAAAKVAQQGLKSVAMRQLAIAAIREAGEKHARDLAELAVSETGMGRVEDKFAKNVAQARGTPGVECLSPQVLTGDNGLTLIENAPWGVVASVTPSTNPAATVINNAISLIAAGNSVIFAPHPAAKKVSQRAITLLNQAIVAAGGPENLLVTVANPDIETAQRLFKFPGIGLLVVTGGEAVVEAARKHTNKRLIAAGAGNPPVVVDETADLARAAQSIVKGASFDNNIICADEKVLIVVDSVADELMRLMEGQHAVKLTAEQAQQLQPVLLKNIDERGKGTVSRDWVGRDAAKIAAAIGLNVPQETRLLFVETTAEHPFAVTELMMPVLPVVRVANVADAIALAVKLEGGCHHTAAMHSRNIENMNQMANAIDTSIFVKNGPCIAGLGLGGEGWTTMTITTPTGEGVTSARTFVRLRRCVLVDAFRIV

SEQ ID NO: 7 - protein, 512 aa, Escherichia coli
MTNNPPSAQIKPGEYGFPLKLKTRYDNFIGGEWVAPADGEYYQNLTPVTGQLLCEVASSGKRDIDLALDAAHKVKDKWAHTSVQDRAAILFKIADRMEQNLELLATAETWDNGKPIRETSAADVPLAIDHFRYFASCIRAQEGGISEVDSETVAYHFHEPLGVVGQIIPWNFPLLMASWKMAPALAAGNCVVLKPARLTPLSVLLLMEIVGDLLPPGVVNVVNGAGGVIGEYLATSKRIAKVAFTGSTEVGQQIMQYATQNIIPVTLELGGKSPNIFFADVMDEEDAFFDKALEGFALFAFNQGEVCTCPSRALVQESIYERFMERAIRRVESIRSGNPLDSVTQMGAQVSHGQLETILNYIDIGKKEGADVLTGGRRKLLEGELKDGYYLEPTILFGQNNMRVFQEEIFGPVLAVTTFKTMEEALELANDTQYGLGAGVWSRNGNLAYKMGRGIQAGRVWTNCYHAYPAHAAFGGYKQSGIGRETHKMMLEHYQQTKCLLVSYSDKPLGLF

SEQ ID NO: 8 - protein, 474 aa, Escherichia coli
MQHKLLINGELVSGEGEKQPVYNPATGDVLLEIAEASAEQVDAAVRAADAAFAEWGQTTPKVRAECLLKLADVIEENGQVFAELESRNCGKPLHSAFNDEIPAIVDVFRFFAGAARCLNGLAAGEYLEGHTSMIRRDPLGVVASIAPWNYPLMMAAWKLAPALAAGNCVVLKPSEITPLTALKLAELAKDIFPAGVINILFGRGKTVGDPLTGHPKVRMVSLTGSIATGEHIISHTASSIKRTHMELGGKAPVIVFDDADIEAVVEGVRTFGYYNAGQDCTAACRIYAQKGIYDTLVEKLGAAVATLKSGAPDDESTELGPLSSLAHLERVSKAVEEAKATGHIKVITGGEKRKGNGYYYAPTLLAGALQDDAIVQKEVFGPVVSVTPFDNEEQVVNWANDSQYGLASSVWTKDVGRAHRVSARLQYGCTWVNTHFMLVSEMPHGGQKLSGYGKDMSLYGLEDYTVVRHVMVKH

SEQ ID NO: 9 - protein, 499 aa, Escherichia coli
MTEPHVAVLSQVQQFLDRQHGLYIDGRPGPAQSEKRLAIFDPATGQEIASTADANEADVDNAVMSAWRAFVSRRWAGRLPAERERILLRFADLVEQHSEELAQLEPLEQGKSIAISRAFEVGCTLNWMRYTAGLTTKIAGKTLDLSIPLPQGARYQAWTRKEPVGVVAGIVPWNFPLMIGMWKVMPALAAGCSIVIKPSETTPLTMLRVAELASEAGIPDGVFNVVTGSGAVCGAALTSHPHVAKISFTGSTATGKGIARTAADRLTRVTLELGGKNPAIVLKDADPQWVIEGLMTGSFLNQGQVCAASSRIYIEAPLFDTLVSGFEQAVKSLQVGPGMSPVAQINPLVSRAHCGKVCSFLDDAQAQQAELIRGSNGPAGEGYYVAPTLVVNPDAKLRLTREEVFGPVVNLVRVADGEEALQLANDTEYGLTASVWTQNLSQALEYSDRLQAGTVWVNSHTLIDANLPFGGMKQSGTGRDFGPDWLDGWCETKSVCVRY

SEQ ID NO: 10 - protein, 482 aa, Escherichia coli
MKLNDSKLFRQQALINGEWLDANNGEVIDVTNPANGDKLGSVPKMGADETRAAIDAANRALPAWRALTAKERANILRNWFNLLMEHQDDLARLMTLEQGKPLAEAKGEISYAASFIEWFAEEGKRIYGDTIPGHQADKRLIVIKQPIGVTAAITPWNFPAAMITRKAGPALAAGCTMVLKPASQTPFSALALAELAIRAGIPAGVFNVVTGSAGAVGNELTSNPLVRKLSFTGSTEIGRQLMEQCAKDIKKVSLELGGNAPFIVFDDADLDKAVEGALSSKFRNAGQTCVCANRLYVQDGVYDRFAEKLQQAVSKLHIGDGLEKGVTIGPLIDEKAVAKVEEHIADALEKGARVVCGGKADERGGNFFQPTILVDVPANAKVSKEETFGPLAPLFRFKDEADVIAQANDTEFGLAAYFYARDLSRVFRVGEALEYGIVGINTGIISNEVAPFGGIKASGLGREGSKYGIEDYLEIKYMCIGL

SEQ ID NO: 11 - protein, 406 aa, Mycobacterium gastri
MKLQVAIDLLSTEAALELAGKVAEYVDIIELGTPLIEAEGLSVITAVKKAHPDKIVFADMKTMDAGELEADIAFKAGADLVTVLGSADDSTIAGAVKAAQAHNKGVVVDLIGIEDKATRAQEVRALGAKFVEMHAGLDEQAKPGFDLNGLLAAGEKARVPFSVAGGVKVATIPAVQKAGAEVAVAGGAIYGAADPAAAAKELRAAIAMTQAAEADGAVKVVGDDITNNLSLVRDEVADTAAKVDPEQVAVLARQIVQPGRVFVAGAGRSGLVLRMAAMRLMHFGLTVHVAGDTTTPAISAGDLLLVASGSGTTSGVVKSAETAKKAGARIAAFTTNPDSPLAGLADAVVIIPAAQKTDHGSHISRQYAGSLFEQVLFVVTEAVFQSLWDHTEVEAEELWTRHANLE

(19) United States
(12) Patent Application Publication
(10) Pub. No.: US 2012/0052542 A1
(43) Pub. Date: Mar. 1, 2012

(54) Reducing Carbon Dioxide Production and Increasing Ethanol Yield During Microbial Ethanol Fermentation

(75) Inventors: Barry Marrs, Kennett Square, PA (US); Brian M. Swalla, Centreville, MD (US)

(73) Assignee: Athena Biotechnologies, Inc., Newark, DE (US)

(21) Appl. No.: 13/254,131

(22) PCT Filed: Mar. 10, 2010
(86) PCT No.: PCT/US10/26803

§ 371 Date: Nov. 15, 2011

Related U.S. Application Data
(60) Provisional application No. 61/158,881, filed on Mar. 10, 2009.

(51) Int. Cl. C12P 7/16; C12N 1/11; C12N 1/21; C12N 1/19; C12N 1/13; C12P 7/06; C12N 1/15

(52) U.S. Cl. 435/160; 435/161; 435/258.1; 435/254.11; 435/254.2; 435/257.2; 435/252.3

(57) ABSTRACT

The present invention provides compositions and methods for producing ethanol wherein the amount of CO₂ by-product is reduced during the fermentation process. The invention includes the use of oxidized lignin during the fermentation process.

### BACKGROUND OF THE INVENTION

Plant biomass is the most abundant source of carbohydrate in the world due to the lignocellulosic materials that comprise the cell walls of plants. Plant cell walls are divided into two classes, primary cell walls and secondary cell walls. The primary cell wall provides structure for expanding cells and comprises three major polysaccharides (cellulose, pectin, and hemicellulose) and one group of glycoproteins. The secondary cell wall, which is produced after the cell has finished growing, also contains polysaccharides and is strengthened through polymeric lignin covalently cross-linked to hemicellulose. Hemicellulose and pectin are typically found in abundance in the secondary cell wall, but cellulose is the predominant polysaccharide and the most abundant source of carbohydrates.

Lignocellulose is a complex substrate comprising a mixture of carbohydrate polymers (namely cellulose and hemicellulose) and lignin. The conversion of lignocellulosic biomass into ethanol relies mainly on the efficient separation of these cell wall components to allow the hydrolysis of the carbohydrates polymer into fermentable sugars. Most of the processes using high temperature or pressure with acid, caustic or organic solvent, are able to provide a cellulose substrate that can be chemically or enzymatically converted into fermentable glucose (Wyman et al. (2005) Bioresource Technology 96:2026-2032; Mosier et al. (2005) Bioresource Technology 96:673-86). In general, the yield and hydrolysis rate of cellulose increases when biomass is fractionated under conditions of high temperature and extremes of pH. Under these severe conditions, however, the overall carbohydrate recovery is often compromised due to extensive degradation of the hemicellulose sugars (mainly xylose in hardwood), which comprise a significant fraction of the lignocellulosic feedstock. Also, the degradation products generated by extensive hydrolysis (phenol, furans and carboxylic acid) can potentially inhibit further fermentation steps (Palmquist et al. (1999) Biotechnol. Bioeng. 63(1):46-55; Klinke et al. (2004) Appl. Microbiol. Biotechnol. 66:10-26).

Ethanol provides a favorable alternative to the use of fossil fuels for energy generation, and increased use of ethanol for fuel could reduce dependence on fossil fuels as well as decrease the accumulation of carbon dioxide in the atmosphere. In the United States, biological production of ethanol, principally by fermentation of grain starches and sugars by yeast, is over four billion liters per year. However, cellulosic biomass potentially provides a far more abundant source of ethanol. Cellulosic biomass represents the greatest carbohydrate resource on earth, and is fixed photosynthetically at a rate of about 10¹¹ tons per year globally.

Conversion of cellulosic biomass to ethanol requires that the polysaccharides of the biomass first be hydrolyzed to fermentable monosaccharides. Cellulose is a polymer of glucose units, and, while hydrolysis of cellulose is more difficult than hydrolysis of starches, hydrolysis of cellulose yields glucose that is readily fermented by yeasts such as Saccharomyces cerevisiae and Kluyveromyces marxianus. However, cellulosic biomass comprises, in addition to cellulose, more complex and heterogeneous polymers collectively known as hemicellulose. Unlike cellulose, hemicellulose contains saccharides besides glucose—principally the pentose xylose, as well as the pentose arabinose and the hexoses glucose, galactose, and mannose. The pentose content of some cellulosic biomass may reach as high as 35% of the total carbohydrate content (see Rosenberg, Enzyme Microbiol Technol 2:185-193 (1980)). Moreover, in many industrial processes, hemicellulose is hydrolyzed to monosaccharides more efficiently than cellulose. Thus, 35-50% of the fermentable sugars obtained by enzymatic or chemical hydrolysis of cellulosic materials may be derived from hemicellulose, and much of this sugar may be in the form of xylose or arabinose (Harris et al., USDA Forest Products Laboratory General Technical Report FPL-45 (1985)).

Microorganisms produce a diverse array of fermentation products. These products include organic acids, such as lactate, acetate, succinate and butyrate, as well as neutral products such as ethanol, butanol, acetone and butanediol. Indeed, the diversity of fermentation products from bacteria has led to their use as a primary determinant in taxonomy. See, for example, Bergey's Manual of Systematic Bacteriology, Williams & Wilkins Co., Baltimore (1984). The microbial production of these fermentation products, by a variety of fermentation culture methods including, adhered or suspended, and batch or continuous, forms the basis of many economically successful applications of biotechnology, including the production of dairy products, meats, beverages and fuels. In recent years, many advances have been made in the field of biotechnology as a result of new technologies which enable researchers to selectively modify the genetic makeup of some microorganisms.

Many bacteria have the natural ability to metabolize simple sugars into a mixture of acidic and neutral fermentation products via the process of glycolysis. Glycolysis is the series of enzymatic steps whereby the six carbon glucose molecule is broken down, via multiple intermediates, into two molecules of the three carbon compound pyruvate. The glycolytic pathways of many bacteria produce pyruvate as a common intermediate. Subsequent metabolism of pyruvate results in a net production of NADH and ATP as well as waste products commonly known as fermentation products. Under aerobic conditions, approximately 95% of the pyruvate produced from glycolysis is consumed in a number of short metabolic pathways which act to regenerate NAD⁺ via oxidative metabolism, where NADH is typically oxidized by donating hydrogen equivalents via a series of steps to oxygen, thereby forming water, an obligate requirement for continued glycolysis and ATP production.

Under anaerobic conditions, most ATP is generated via glycolysis. Additional ATP can also be regenerated during the production of organic acids such as acetate, NAD⁺ is regenerated from NADH during the reduction of organic substrates such as pyruvate or acetyl CoA. Therefore, the fermentation products of glycolysis and pyruvate metabolism include organic acids, such as lactate, formate and acetate as well as neutral products such as ethanol.

The majority of facultatively anaerobic bacteria do not produce high yields of ethanol either under aerobic or anaerobic conditions. Most facultative anaerobes metabolize pyruvate aerobically via pyruvate dehydrogenase (PDH) and the tricarboxylic acid cycle (TCA). Under anaerobic conditions, the main energy pathway for the metabolism of pyruvate is via pyruvate-formate-lyase (PFL) pathway to give formate and acetyl-CoA. Acetyl-CoA is then converted to acetate, via phosphotransacetylase (PTA) and acetate kinase (AK) with the co-production of ATP, or reduced to ethanol via acetaldehyde dehydrogenase (AcDH) and alcohol dehydrogenase (ADH). In order to maintain a balance of reducing equivalents, excess NADH produced from glycolysis is re-oxidized to NAD⁺ by lactate dehydrogenase (LDH) during the reduction of pyruvate to lactate. NADH can also be re-oxidized by AcDH and ADH during the reduction of acetyl-CoA to ethanol but this may be a minor reaction in cells with a functional LDH. Theoretical yields of ethanol are therefore not achieved since most acetyl CoA is converted to acetate to regenerate ATP and excess NADH produced during glycolysis is oxidized by LDH.

Ethanologenic organisms, such as Zymomonas mobilis and yeast, are capable of a second type of anaerobic fermentation commonly referred to as an alcoholic fermentation in which pyruvate is metabolized to acetaldehyde and CO₂ by pyruvate decarboxylase (PDC). Acetaldehyde is then reduced to ethanol by ADH regenerating NAD Alcoholic fermentation results in the metabolism of 1 molecule of glucose to two molecules of ethanol and two molecules of CO₂. The genes that encode both of these enzymes in Z. mobilis have been isolated, cloned and expressed recombinantly in hosts capable of producing high yields of ethanol via the synthetic route described above. For example; U.S. Pat. No. 5,000,000 and Ingram et al (1997) Biotechnology and Bioengineering 58, Nos. 2 and 3 have shown that the genes encoding both PDC (pdc) and ADH (adh) from Z. mobilis can be incorporated into a “pet” operon which can be used to transform Escherichia coli strains resulting in the production of recombinant E. coli capable of co-expressing the Z. mobilis pdc and adh. This results in the production of a synthetic pathway re-directing E. coli central metabolism to produce ethanol from pyruvate during growth under both aerobic and anaerobic conditions. Similarly, U.S. Pat. No. 5,554,520 discloses that pdc and adh from Z. mobilis can both be integrated via the use of a pet operon to produce Gram negative recombinant hosts, including Erwina, Klebsiella and Xanthomonas species, each of which expresses the heterologous genes of Z. mobilis resulting in high yield production of ethanol via a synthetic pathway from pyruvate to ethanol.

U.S. Pat. No. 5,482,846 discloses the simultaneous transformation of Gram positive Bacillus sp with heterologous genes which encode both the PDC and ADH enzymes so that the transformed bacteria produce ethanol as a primary fermentation product. There is no suggestion that the bacteria may be transformed with the pdc gene alone.

Production of ethanol from cellulosic biomass via microbial fermentation requires the co-production of carbon dioxide (or other oxidized by-product) to maintain the required reduction-oxidation (redox) balance among fermentation products. Redox describes all chemical reactions in which atoms have their oxidation number (oxidation state) changed. This can be either a simple redox process such as the oxidation of carbon to yield carbon dioxide or the reduction of carbon by hydrogen to yield methane (CH₄), or it can be a complex process such as the oxidation of sugar in the human body through a series of very complex electron transfer processes. The term redox comes from the two concepts of reduction and oxidation. Oxidation describes the loss of electrons/hydrogen or gain of oxygen/increase in oxidation state by a molecule, atom or ion. Reduction describes the gain of electrons/hydrogen or a loss of oxygen/decrease in oxidation state by a molecule, atom or ion.

Carbon dioxide is an undesirable industrial reaction byproduct due to its environmental impact as a “greenhouse gas”, and thus methods to reduce its production in large-scale industrial processes are valuable. The present invention satisfies the need in the art for a more efficient method of producing ethanol via microbial fermentation and reducing or eliminating the production of carbon dioxide during the process.

### BRIEF SUMMARY OF THE INVENTION

The invention provides a method of reducing production of CO₂ in a fermentation process of producing an alcohol. In one embodiment, the method comprises incubating a microorganism in a culture medium, wherein the culture medium comprises fermentable and non-fermentable portions, and further wherein the non-fermentable portion of the culture medium can be oxidized by the microorganism thereby minimizing the need for oxidation of the fermentable portion.

In one embodiment, the alcohol is ethanol or butanol.

In one embodiment, the yield of ethanol production is increased.

In one embodiment, the non-fermentable portion comprises lignin.

In one embodiment, the fermentable portion comprises carbohydrates.

In one embodiment, the microorganism has been modified to eliminate production of CO₂ from formate.

In one embodiment, the modification is the inactivation of formate-hydrogen lyase (FHL) and formate dehydrogenase (FDH).

In one embodiment, the microorganism is modified to express a component of a pathway that converts formate to formaldehyde.

In one embodiment, the microorganism has been modified to express formate reductase (FMR).

In one embodiment, the microorganism has been modified to assimilate carbon from a one-carbon compound.

In one embodiment, the modification comprises expressing a component of the ribulose monophosphate pathway.

In one embodiment, the component of the ribulose monophosphate pathway is hexulose phosphate synthase (HPS) and phosphohexulose isomerase (PHI).

In one embodiment, the microorganism has been modified to express an oxidoreductase enzyme wherein the enzyme catalyzes the oxidation of the non-fermentable portion of the culture medium to support the conversion of oxidized biological cofactors to reduced cofactors.

In one embodiment, the enzyme is able to oxidize lignin.

In one embodiment, the enzyme is phosphate dehydrogenase (PTDH).

In one embodiment, the microorganism is cultured in an electrochemical bioreactor.

In one embodiment, the microorganism is modified to produce an electron shuttle that is secreted outside the microorganism, and wherein said electron shuttle is capable of transferring electrons to the cell to support the intracellular conversion of oxidized biological cofactors to reduced cofactors.

In one embodiment, the electron shuttle is a small molecule.

In one embodiment, the electron shuttle is a protein.

In one embodiment, the microorganism has been modified to reduce or eliminate production of carbon dioxide from pyruvate by inactivating pyruvate decarboxylase (PDC).

In one embodiment, the microorganism has been modified to reduce or eliminate production of carbon dioxide from pyruvate by inactivating pyruvate-ferredoxin oxidoreductase (PFO).

In one embodiment, the microorganism has been modified to reduce or eliminate production of carbon dioxide from pyruvate by inactivating pyruvate dehydrogenase.

In one embodiment, the microorganism has been modified to reduce or eliminate production of any one or more of carbon dioxide from pyruvate by inactivating pyruvate decarboxylase (PDC), carbon dioxide from pyruvate by inactivating pyruvate-ferredoxin oxidoreductase (PFO), or carbon dioxide from pyruvate by inactivating pyruvate dehydrogenase, further wherein the microorganism has been modified to enable conversion of pyruvate to acetyl-CoA for production of formate instead of carbon dioxide.

In one embodiment, the modification to enable conversion of pyruvate to acetyl-CoA comprises expression of pyruvate-formate lyase (PFL).

In one embodiment, the microorganism is modified to prevent production of carbon dioxide from formate by inactivating formate dehydrogenase (FDH).

In one embodiment, the microorganism is modified to express an enzyme that converts formate to formaldehyde.

In one embodiment, the enzyme is formate reductase.

In one embodiment, the microorganism has been modified to utilize the ribulose monophosphate pathway to convert three formaldehyde molecules into glyceraldehyde-3 -phosphate.

In one embodiment, the microorganism has been modified to utilize the serine pathway to assimilate carbon from formaldehyde and carbon dioxide into 3-phosphoglycerate.

In one embodiment, the microorganism has been modified to express an oxidoredutase enzyme wherein the enzyme catalyzes the oxidation of the non-fermentable portion of the culture medium to support the conversion of oxidized biological cofactors to reduced cofactors.

In one embodiment, the enzyme is able to oxidize lignin.

In one embodiment, the enzyme is phosphate dehydrogenase (PTDH).

In one embodiment, the microorganism is cultured in an electrochemical bioreactor.

In one embodiment, the microorganism is modified to produce an electron shuttle that is secreted outside the microorganism, and wherein said electron shuttle is capable of transferring electrons to the cell to support the intracellular conversion of oxidized biological cofactors to reduced cofactors.

In one embodiment, the microorganism has been modified to utilize the Calvin cycle to convert six carbon dioxide molecules into fructose-6-phosphate.

In one embodiment, the lignin is modified in either a chemical or biological process to be more oxidizable by the microorganism.

The invention provides a microorganism modified to permit the reduced production of CO₂ in a fermentation process, wherein said modification is the activation of an oxidoreductase enzyme, wherein said enzyme is capable of catalyzing the oxidation of lignin.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is schematic of a representative strategy for cloning formate reductase genes.

FIG. 2 is a schematic of a representative strategy for cloning ribulose monophosphate pathway genes.

FIG. 3 is a schematic of a representative strategy for cloning phosphite dehydrogenase genes.

FIG. 4A is a schematic depicting homoethanol fermentation as found in yeast, Zymomonas mobilis and certain engineered E. coli strains, in which one molecule of CO₂ is produced for every molecule of ethanol. Enzyme reactions include: (a) glycolysis (several enzymes), (b) pyruvate decarboxylase (PDC, which is native in yeast and Z. mobilis, and has been engineered into E. coli), and (c) alcohol dehydrogenase.

FIG. 4B is a schematic depicting a simplified view of the engineered metabolic pathways in a homoethanologen microbe engineered to eliminate CO₂ production and increase ethanol production. PDC (FIG. 4A, reaction b) is inactivated (or not introduced in the case of E. coli), and the pathway comprising (d) pyruvate-formate lyase (PFL) and (e) acetaldehyde dehydrogenase (ALDH) is introduced, except in E. coli where the PFL-ALDH pathway is native. The formate produced by PFL is shunted into a pathway introduced from methylotrophic bacteria, the ribulose monophosphate pathway (RuMP), which converts the formate to sugars and ultimately to ethanol in the organism. The PFL reaction and the RuMP pathway each require additional NADH, which can be generated by an “external reductant”.

FIG. 5 is a schematic depicting conversion of formaldehyde into glyceraldehyde-3-phosphate (G3P) by the ribulose monophosphate (RuMP) pathway when expressed in E. coli. Two key RuMP enzymes that are cloned and expressed are: hexulose phosphate synthase (HPS; reaction 1); and phosphohexulose isomerase (PHI; reaction 2). The remaining reactions are catalyzed by native E. coli enzymes. Ribulose 5-P cofactor is regenerated by multiple sugar-rearrangements catalyzed by pentose phosphate and glycolysis pathway enzymes (reactions 3-5). Dihydroxy-acetone-phosphate is converted into G3P by triosephosphate isomerase (reaction 6). G3P is a glycolysis intermediate that can be converted into pyruvate, and ultimately, ethanol.

FIG. 6 is a schematic depicting homoethanol fermentation by an E. coli strain engineered to oxidize a auxiliary substrate (in this example, potassium phosphite; K₂HPO₃) and assimilate one-carbon compounds, thereby (1) eliminating CO₂ production and (2) increasing ethanol yield. Key innovations are highlighted in gray shaded boxes. Net chemical products are shown in black outlined boxes. Additional NADH consumed by the system is shown in black “clouds”. Box A: Oxidoreductase enzyme (phosphite dehydrogenase; reaction “h”) oxidizes phosphite to phosphate coupled with reduction of NAD+ to NADH. Box B: Pyruvate-formate lyase (PFL; reaction “d”) produces formate instead of CO₂ from pyruvate. Two additional NADH from phosphite oxidation are required to convert acetyl-CoA to acetaldehyde (reaction “e”) for homoethanol production. Box C: Formate is reduced to formaldehyde by the formate reductase (reaction “f”) using NADH from phosphite oxidation. Note that one formate molecule is cycled. Box D: Formaldehyde is assimilated into glyceraldehyde-3-P via the ribulose monophosphate pathway (RuMP), obtained from a methylotrophic bacterium. The glyceraldehyde-3-P produced in Box D is converted to ethanol through glycolysis and the reactions in Box B, which requires one additional NADH from phosphite oxidation. Collectively, six (6) NADH from phosphite oxidation enable three ethanol molecules to be made from each glucose molecule. Although not shown, both 6-carbon (e.g. glucose) and 5-carbon sugars (e.g. arabinose, xylose) may be metabolized in this scheme.

FIG. 7 is a schematic representation of the 3-Chamber Electrochemical Bioreactor of Hwang et al. (2008, Biotechnol Bioprocess Eng 13: 677-682).

### DETAILED DESCRIPTION OF THE INVENTION

The present invention provides compositions and methods for reducing or eliminating production of carbon dioxide during the fermentation process of producing ethanol or other products from biomass.

Prior art fermentation processes waste a significant portion of sugar to make CO₂ instead of a desirable product because the microorganism must produce CO₂ in order to maintain the redox balance. Accordingly, the invention relates to the use of an auxiliary source of electrons wherein the sugar in the feedstock is not oxidized to CO₂ thereby making the sugar more available for conversion into a desirable product. Thus, the invention includes both reducing CO₂ production and increasing yield of a desirable fermentation product by using an auxiliary source of electrons as a means to maintain the redox balance.

The invention also encompasses compositions and methods useful for oxidizing lignin in biomass instead of oxidizing part of sugar starting source. In some instances, the invention includes using lignin contained in cellulosic biomass feedstocks as a source of electrons for the reduction of pyruvate to ethanol.

The invention also encompasses compositions and methods useful for oxidizing reduced agents instead of oxidizing part of sugar starting source. In some instances, the invention includes using reduced chemicals as a source of electrons for the reduction of pyruvate to ethanol. In some instances, the invention includes enzymatic oxidation of reduced agents as a source of electrons for the reduction of pyruvate to ethanol.

The invention also encompasses compositions and methods useful for using electric power instead of oxidizing part of sugar starting source. In some instances, the invention includes using electric power as a source of electrons for the reduction of pyruvate to ethanol.

The invention also provides compositions and methods for increasing the yield of ethanol produced from the fermentation of biomass by directing the flow of carbon atoms previously utilized for carbon dioxide production into a biosynthetic pathway to produce additional ethanol.

In one embodiment, an organism is genetically modified to enable the organism to convert all or substantially all of the sugars in the feedstock (for example both hexoses and pentoses), into ethanol by utilizing an auxiliary source of electrons, such as oxidizing an external reductant, thereby not giving rise to CO₂, or giving rise to much less CO₂ compared to prior art processes.

The genetic modification allows for less CO₂ production and more ethanol produced from each unit of feedstock. In some instances, an electric current is used as the external reductant. In other instances, lignin can be used as a source of external reductant. In still other instances, reduced chemicals are used as the source of electrons.

### Definitions

The articles “a” and “an” are used herein to refer to one or to more than one (i.e. to at least one) of the grammatical object of the article. By way of example, “an element” means one element or more than one element.

As used herein the term “alcohol dehydrogenase” or “ADH” is intended to include the enzyme capable of converting acetaldehyde into an alcohol, advantageously, ethanol. In some instances, ADH utilizes the electrons from NADH to reduce acetaldehyde to ethanol. In some instances, a type of ADH is able to catalyze the oxidation of α-hydroxyl groups in lignin to ketones with the coincident reduction of NAD+ to NADH.

A “conservative substitution” is the substitution of an amino acid with another amino acid with similar physical and chemical properties. In contrast, a “nonconservative substitution” is the substitution of an amino acid with another amino acid with dissimilar physical and chemical properties.

The term “decarboxylase activity” is intended to include the ability of a polypeptide to enzymatically convert pyruvate into acetaldehyde. Typically, the activity of a selected polypeptide encompasses the total enzymatic activity associated with the produced polypeptide, comprising, e.g., the superior substrate affinity of the enzyme, thermostability, stability at different pHs, or a combination of these attributes.

The term “ethanologenic” is intended to include the ability of a microorganism to produce ethanol from a carbohydrate as a primary fermentation product. The term is intended to include naturally occurring ethanologenic organisms, ethanologenic organisms with naturally occurring or induced mutations, and ethanologenic organisms which have been genetically modified.

The terms “fermenting” and “fermentation” are intended to include the enzymatic process (e.g., cellular or acellular, e.g., a lysate or purified polypeptide mixture) by which ethanol is produced from a carbohydrate, in particular, as a primary product of fermentation.

The term “gram-negative bacterial cell” is intended to include the art recognized definition of this term. Typically, Gram-negative bacteria include Gluconobacter, Rhizobium, Bradyrhizobium, Alcaligenes, Rhodobacter, Rhodococcus, Azospirillum, Rhodospirillum, Sphingomonas, Burkholderia, Desulfomonas, Geospirillum, Succinomonas, Aeromonas, Shewanella, Halochromatium, Citrobacter, Escherichia, Klebsiella, Zymomonas (e.g., Zymomonas mobilis), Zymobacter (e.g., Zymobacter palmae), and Acetobacter (e.g., Acetobacter pasteurianus).

The term “gram-positive bacteria” is intended to include the art recognized definition of this term. Typically, Gram-positive bacteria include Fibrobacter, Acidobacter, Bacteroides, Sphingobacterium, Actinomyces, Corynebacterium, Nocardia, Rhodococcus, Propionibacterium, Bifidobacterium, Bacillus, Geobacillus, Paenibacillus, Sulfobacillus, Clostridium, Anaerobacter, Eubacterium, Streptococcus, Lactobacillus, Leuconostoc, Enterococcus, Lactococcus, Thermobifida, Cellulomonas, and Sarcina (e.g. Sarcina ventriculi).

As used herein, the terms “gene” and “recombinant gene” refer to nucleic acid molecules comprising an open reading frame encoding a polypeptide.

As used herein, the term “genetically engineered” refers to a modification of the inherent genetic material of a microorganism (e.g., one or more of the deletion, addition, or mutation of one or more nucleic acid residues within the genetic material), additional of exogenous genetic material to a microorganism (e.g., stable plasmid, integrating plasmid, naked genetic material, among other things), causing the microorganism to alter its genetic makeup due to external or internal signaling (e.g., environmental pressures, chemical pressures, among other things), or any combination of these or similar techniques for altering the overall genetic makeup of the organism.

The term “glycolysis” refers to a pathway for the conversion of a glucose molecule into two pyruvate molecules within the microorganism, which in the microorganism is also associated with net production of two ATP molecule and two NAD(P)H molecule. Glycolysis may also be referred to as the “Embden-Meyerhof pathway”.

The term “TCA cycle” as used herein refers to a pathway wherein the acetate is converted in a cyclical manner, into carbon dioxide and NAD(PH). TCA cycle may also be referred to as “tricarboxylic acid cycle” or “Krebs cycle.”

As used herein, the term “pathway” refers to a biological process including two or more enzymatically controlled chemical reactions by which a substrate is converted into a product.

As used herein, “homology” is used synonymously with “identity.”

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

A “polynucleotide” means a single strand or parallel and anti-parallel strands of a nucleic acid. Thus, a polynucleotide may be either a single-stranded or a double-stranded nucleic acid.

The term “nucleic acid” typically refers to a large polynucleotide.

The term “oligonucleotide” typically refers to short a polynucleotide, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

“Recombinant polynucleotide” refers to a polynucleotide having sequences that are not naturally joined together. An amplified or assembled recombinant polynucleotide may be included in a suitable vector, and the vector can be used to transform a suitable host cell. A recombinant polynucleotide may serve a non-coding function (e.g., promoter, enhancer, origin of replication, ribosome-binding site, etc.) as well.

A “recombinant polypeptide” is one which is produced upon expression of a recombinant polynucleotide.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a condition-specific manner.

“Mutants,” “derivatives,” and “variants” of a polypeptide (or of the DNA encoding the same) are polypeptides which may be modified or altered in one or more amino acids (or in one or more nucleotides) such that the peptide (or the nucleic acid) is not identical to the wild-type sequence, but has homology to the wild type polypeptide (or the nucleic acid).

A “mutation” of a polypeptide (or of the DNA encoding the same) is a modification or alteration of one or more amino acids (or in one or more nucleotides) such that the peptide (or nucleic acid) is not identical to the sequences recited herein, but has homology to the wild type polypeptide (or the nucleic acid).

As used herein, a “mutant form” of a gene is a gene which has been altered, either naturally or artificially, changing the base sequence of the gene, which results in a change in the amino acid sequence of an encoded polypeptide. The change in the base sequence may be of several different types, including changes of one or more bases for different bases, small deletions, and small insertions. Mutations may also include transposon insertions that lead to attenuated activity, i.e., by resulting in expression of a truncated protein. By contrast, a normal form of a gene is a form commonly found in a natural population of an organism. Commonly a single form of a gene will predominate in natural populations. In general, such a gene is suitable as a normal form of a gene; however, other forms which provide similar functional characteristics may also be used as a normal gene.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

A “portion” of a polynucleotide means at least about twenty sequential nucleotide residues of the polynucleotide. It is understood that a portion of a polynucleotide may include every nucleotide residue of the polynucleotide.

The term “modulate,” as used herein, refers to any change from the present state. The change may be an increase or a decrease. For example, the activity of an enzyme may be modulated such that the activity of the enzyme is increased from its current state. Alternatively, the activity of an enzyme may be modulated such that the activity of the enzyme is decreased from its current state.

As the term is used herein, “population” refers to two or more cells.

The term “engineer” refers to any manipulation of a microorganism that result in a detectable change in the microorganism, wherein the manipulation includes but is not limited to inserting a polynucleotide and/or polypeptide heterologous to the microorganism and mutating a polynucleotide and/or polypeptide native to the microorganism. A polynucleotide or polypeptide is “heterologous” to a microorganism if it is not part of the polynucleotides and polypeptides expressed in the microorganism as it exists in nature, i.e., it is not part of the wild-type of that microorganism. A polynucleotide or polypeptide is instead “native” to a microorganism if it is part of the polynucleotides and polypeptides expressed in the microorganism as it exists in nature, i.e., it is part of the wild-type of that microorganism. The term “mutation” as used herein indicates any modification of a nucleic acid and/or polypeptide which results in an altered nucleic acid or polypeptide. Mutations include, for example, point mutations, deletions, or insertions of single or multiple residues in a polynucleotide, which includes alterations arising within a protein-encoding region of a gene as well as alterations in regions outside of a protein-encoding sequence, such as, but not limited to, regulatory or promoter sequences.

The term “enzyme” as used herein refers to any substance that catalyzes or promotes one or more chemical or biochemical reactions, which usually includes enzymes totally or partially composed of a polypeptide, but can include enzymes composed of a different molecule including polynucleotides.

The term “microorganism” is used herein interchangeably with the terms “cell,” “microbial cells” and “microbes” and refers to an organism of microscopic or ultramicroscopic size such as a prokaryotic or a eukaryotic microbial species. The term “prokaryotic” refers to a microbial species which contains no nucleus or other organelles in the cell, which includes but is not limited to Bacteria and Archaea. The term “eukaryotic” refers to a microbial species that contains a nucleus and other cell organelles in the cell, which includes but is not limited to Eukarya such as yeast and filamentous fungi, protozoa, algae, or higher Protista.

The term “oxidoreductase” as used herein refers to an enzyme that catalyzes the transfer of electrons from one molecule (the reductant, also called the hydrogen or electron donor) to another (the oxidant, also called the hydrogen or electron acceptor). Electron donors include carrier molecules such as NADH or NAD(P)H that contain reducing equivalents wherein the term “reducing equivalents” refers to electrons usually generated through oxidation of a substrate during aerobic or anaerobic metabolism that are contained in the carrier molecule. Electron acceptors include the oxidized form of carrier molecules NADH and NADPH, i.e. NAD+ and NADP+. The term “substrate as used herein refers to any substance or compound that is converted or meant to be converted into another compound by the action of an enzyme catalyst.

An “NAD(P)H-requiring oxidoreductase” as used herein refers to an enzyme that catalyzes a reaction involving the transfer of reducing; equivalents directly or indirectly donated by NADH or NADPH. An “NAD(P)H producing oxidoreductase” as used herein refers to an enzyme that catalyzes a reaction involving the transfer of reducing equivalents directly or indirectly donated to an NAD⁺ or NADP³⁰ .

### Description

The present invention relates to methods and compositions for increasing ethanol yield and eliminates or lowers CO₂ as a byproduct of ethanol fermentation by engineering one or more unique metabolic pathways into the production organism. The invention is based on preventing CO₂ production from the organism by supplying reducing power from an auxiliary source (i.e. not from primary fermentable hexose or pentose sugars in the feedstock) to produce ethanol. Supplying a reducing power enables an increased ethanol yield by capturing more carbon and energy from the feedstock in the ethanol product. Accordingly, the invention also provides engineered microbes for ethanol production from a cellulosic and non-cellulosic composition.

Equimolar CO₂ and fermentation products (e.g., ethanol) are produced during homoethanol fermentations to maintain reduction-oxidation (redox) equilibrium in a cell. For example, yeast produce two molecules of ethanol (oxidation state: -4) and two molecules of CO₂ (oxidation state: +4) from each glucose. The present invention relates to discovery that equimolar CO₂ production is not required if a substrate other than fermentable hexose (e.g. glucose) and pentose sugars is available to be oxidized.

The present invention provides compositions and methods for producing a fermentation product from biomass, preferably lignocellulosic material, more preferably cellulose, lignin, and combinations thereof. The invention includes reducing CO₂ production during a fermentation process to make ethanol (or other products from biomass). Decreasing the amount of CO₂ produced is a desirable embodiment of the invention because CO₂ is an undesirable by-product of the fermentation process.

The invention encompasses utilizing an auxiliary source of electrons rather than oxidizing part of the sugar derived from the feedstock. The standard fermentation process used in the art is inefficient because feedstock carbon is consumed to make CO₂ rather than a desired product, because existing microbes must produce an oxidized by-product in order to maintain the redox balance. For example, one third of the carbohydrate carbon is converted into CO₂, and only two thirds go to ethanol in prior art methods. The present invention is based on the ability to eliminate or reduce the production of CO₂ in the fermentation process by improving the efficiency of conversion of biomass to ethanol through the use of an external reductant. In some instances, the auxiliary source of electrons is provided by oxidation of lignin. In other instances, the auxiliary source of electrons is provided by oxidation of reduced chemicals. In other instances, the auxiliary source of electrons is provided by electric power. In some instances, any combination of the auxiliary sources of electrons can be used.

This invention is thus an improvement of the methods used in the art because the feedstock consumed in the fermentation process results in less CO₂ production thereby allowing for the production of more desired products, such as ethanol. That is, a benefit of the invention is that yield can be increased by redirecting the flow of carbon hitherto destined for CO₂ production into a pathway to produce additional fermentation product.

The invention relates to the use of an auxiliary source of electrons wherein the fermentable feedstock sugar is not oxidized to CO₂ to maintain the redox balance thereby making the sugar more available for conversion into a desirable product. Thus, the invention includes both reducing CO₂ production and increasing a desirable fermentation product by utilizing electrons from an auxiliary source as a means to maintain the redox balance. Utilization of electrons from an auxiliary source allows for the microorganism to maintain the redox balance without the requirement of oxidizing the desired carbon source such as glucose for the production of a product such as CO₂. For example, using lignin as a source of electrons for the reduction of pyruvate to ethanol increases the yield of ethanol produced from the fermentation of biomass by directing the flow of carbon atoms previously utilized for carbon dioxide production into a biosynthetic pathway to produce additional ethanol. In some instances, reduced chemicals are oxidized as a source of auxiliary electrons. In other instances, electric power is used as a source of auxiliary electrons. However, the invention should not be limited to any particular auxiliary source of electrons. This is because the invention includes the use of any auxiliary source of electrons known in the art or those to be identified in the future.

### Engineered Microorganism

Many bacteria have the natural ability to metabolize simple sugars into a mixture of acidic and neutral fermentation products, in part, via the process of glycolysis. Glycolysis is the series of enzymatic steps whereby the six carbon glucose molecule is broken down, via multiple intermediates, into two molecules of the three carbon compound pyruvate. The glycolytic pathways of many bacteria produce pyruvate as a common intermediate. Under aerobic conditions, approximately 95% of the pyruvate produced from glycolysis is consumed in a number of short metabolic pathways which act to regenerate NAD+ via oxidative metabolism, where NADH is typically oxidized by donating hydrogen equivalents via a series of steps to oxygen, thereby forming water, an obligate requirement for continued glycolysis and ATP production.

Prior art anaerobic fermentation processes waste a significant portion of sugar to make CO₂ instead of a desirable product because the microorganism must produce CO₂ in order to maintain the redox balance. The present invention comprises an engineered microorganism that is able to reduce production of CO₂ compared to a wild-type microorganism during fermentation because the engineered microorganism is able to utilize electrons from an auxiliary source. In some instances, the engineered microorganism is able to oxidize waste lignin contained in cellulosic biomass feedstocks. In other instances, the engineered microorganism is able to oxidize reduced chemicals added to the fermenter. In other instances, the engineered microorganism is able to utilize electrons provided via electric power. That is, the invention contemplates any engineered microorganism capable of oxidizing electrons from an auxiliary source. An advantage of the engineered microorganism of the invention is that microorganism can produce more fermentation product because the flow of carbon hitherto destined for CO₂ production is redirected into a pathway to produce additional fermentation product. This is because equimolar CO₂ production is not required if a substrate other than fermentable hexose and pentose sugars is available for oxidation.

The auxiliary source of electrons allows the engineered microbe to maintain the redox balance without having the microorganism oxidize a desired carbon source such as glucose. Accordingly, the invention relates to the use of electrons from an auxiliary source wherein the desired sugar is not oxidized to CO₂ to maintain the redox balance thereby making the sugar more available for conversion into a desirable product. Thus, the engineered microorganism is able to both reduce CO₂ production and increase production of a desirable fermentation product by being able to utilize an auxiliary source of electrons rather than oxidize a desired carbon source as a means to maintain the redox balance. The strategy of engineering microorganisms to be able to utilize an auxiliary electron source to prevent oxidation of a desired carbon source to CO₂ can be applied to any existing microorganism used in fermentation as set forth in elsewhere herein. The invention should not be construed to be limited to only modification of microorganisms discussed herein.

Eliminating Production of CO₇ from Formate.

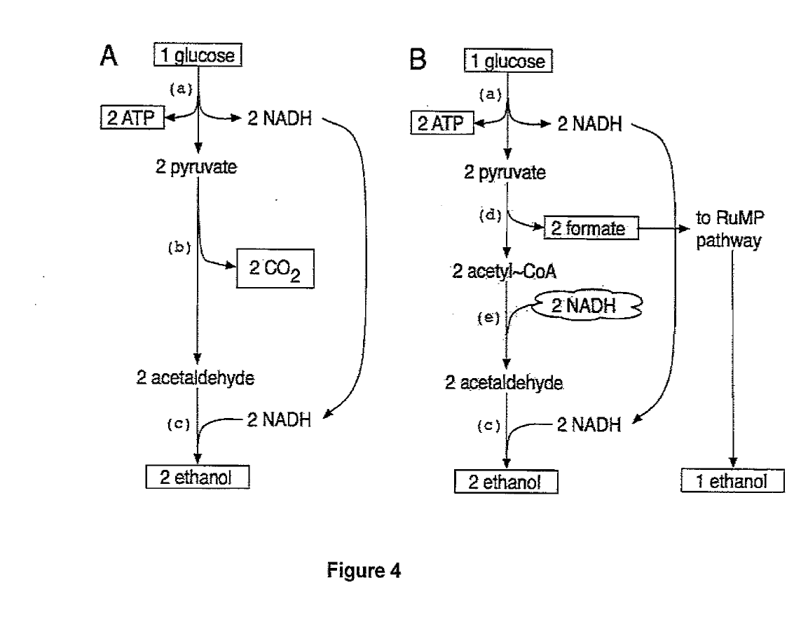

In a typical fermentation catalyzed by the yeast Saccharomyces cerevisiae, glucose is oxidized during glycolysis into two molecules of pyruvate with the co-reduction of two molecules of NAD+ to NADH (FIG. 4A). Pyruvate decarboxylase (PDC) produces one molecule of acetaldehyde and carbon dioxide from each pyruvate. Alcohol dehydrogenase (ADH) utilizes the electrons from NADH to reduce acetaldehyde to ethanol.

In some bacterial fermentations, such as mixed-acid fermentation performed by E. coli, PDC is replaced by three enzymes, pyruvate-formate lyase (PFL), formate dehydrogenase (FDH, which may be part of the formate-hydrogen lyase complex), and acetaldehyde dehydrogenase (ACDH) (FIG. 4B). These enzymes catalyze the conversion of pyruvate to carbon dioxide and acetaldehyde in three discrete steps. First, PFL converts pyruvate and coenzyme-A into formate and acetyl-CoA. FDH then catalyzes oxidation of formate to carbon dioxide (which may be coupled to reduction of NAD+ to NADH). ACDH utilizes the NADH to reduce acetyl-CoA to acetaldehyde and regenerate coenzyme-A. In the bacterial-type pathway, FDH can be inactivated to stop production of carbon dioxide, thereby preventing loss of this carbon from the cell.

In one embodiment, the engineered microbe of the invention used for ethanol production is natively able to produce ethanol using the bacterial pathway comprising pyruvate-formate lyase (PFL) and acetaldehyde dehydrogenase (ACDH). In a preferred embodiment, the microbe is E. coli.

In another embodiment, a microbe used for ethanol production that does not natively express the PFL and ACDH enzymes is engineered to express the bacterial pathway for ethanol production comprising PFL and ACDH. In a preferred embodiment, the microbe is yeast. In a more preferred embodiment, the microbe is a strain of Saccharomyces cerevisiae. In another preferred embodiment, the microbe is a bacterium. In a more preferred embodiment, the microbe is a strain of Zymomonas mobilis.

In one embodiment, the microbe of the invention is engineered to inactivate other competing pathways for ethanol production that produce CO₂ from pyruvate. In a preferred embodiment, one or more of the following enzymes or complexes are inactivated: pyruvate decarboxylase (PDC), pyruvate dehydrogenase (PDH), or pyruvate-ferredoxin oxidoreductase (PFO).

In one embodiment, the microbe of the invention is engineered to inactivate FDH to eliminate production of CO₂ from formate. In one embodiment, the microbe is engineered to inactivate the formate-hydrogen lyase (FHL) complex, which may contain a subunit with FDH activity. In a preferred embodiment, the mutation inactivating FDH affects the fdhF gene.

Conversion of Formate into Ethanol.

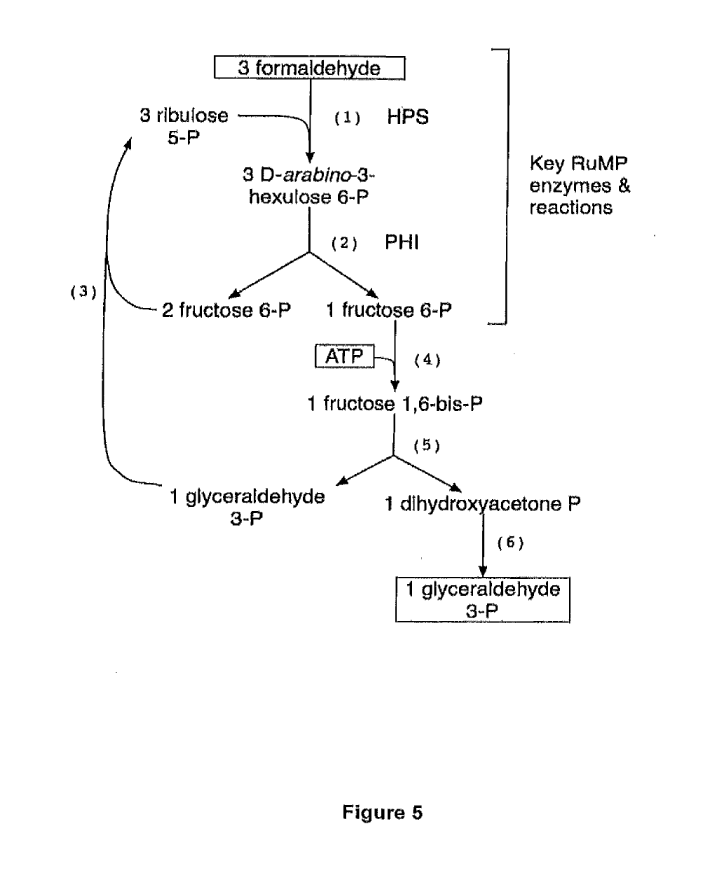

When the cell is engineered with a metabolic pathway that can assimilate one-carbon compounds, and sufficient reducing power is available to the cell, the formate that accumulates due to the lack of FDH activity can be converted into ethanol. For example, an engineered pathway comprising formate reductase (FMR) (Tani et al. Agric Biol Chem, 1978, 42: 63-68; Agric Biol Chem, 1974, 38: 2057-2058) and the ribulose monophosphate (RuMP) pathway, which is found in “type I” methylotrophic bacteria (Lidstrom 2006, Prokaryotes 2: 618-634), can be used and is described elsewhere herein. FMR enzyme reduces formate to formaldehyde coupled with oxidation of the cofactor NAD(P)H to NAD(P)+. The RuMP pathway converts three formaldehyde molecules and one adenosine triphosphate (ATP) into glyceraldehyde-3-phosphate, which is a glycolysis intermediate that can be metabolized to pyruvate and energy, and ultimately ethanol (FIG. 5).

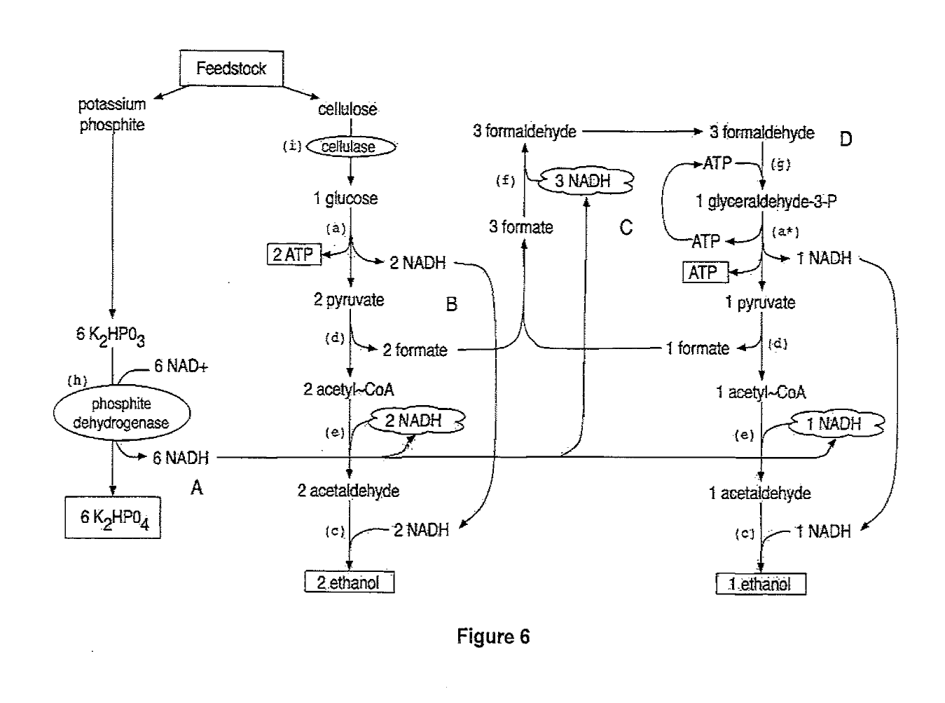

In one embodiment, the microorganism is engineered to take advantage of the scheme shown in FIG. 6, whereby the microorganism is able to convert all or substantially all of the hexose (e.g. glucose) and pentose sugars (e.g. arabinose and xylose) into ethanol, and oxidize an external reductant (shown in FIG. 6 as phosphite, for example), phosphite that does not give rise to CO₂, or gives rise to much less CO₂ compared to prior art processes. The metabolic changes resulting from the genetic modification of the organism allows for less CO₂ and more ethanol production from each unit of feedstock.

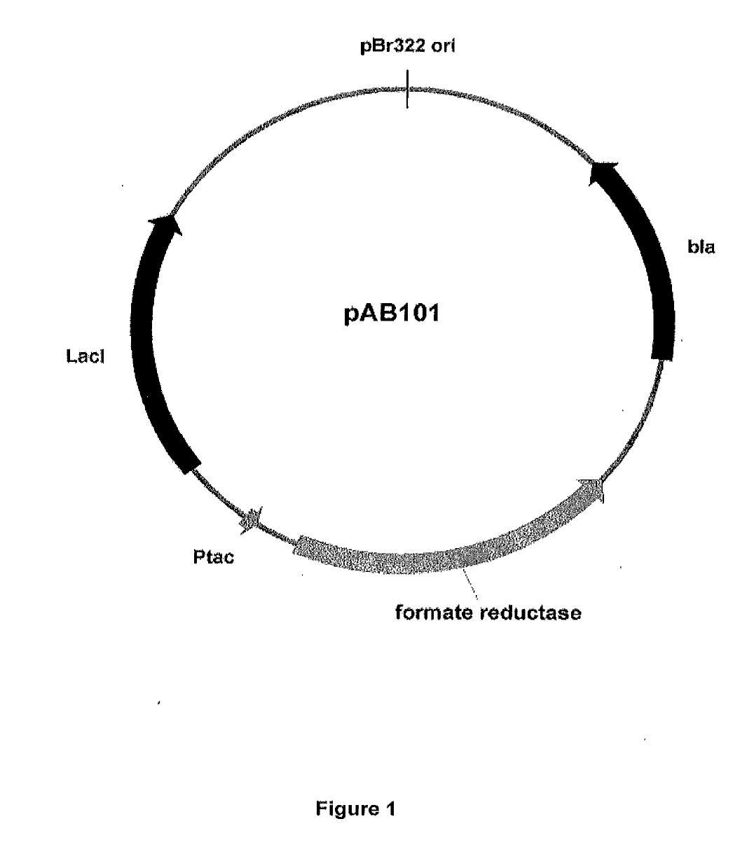

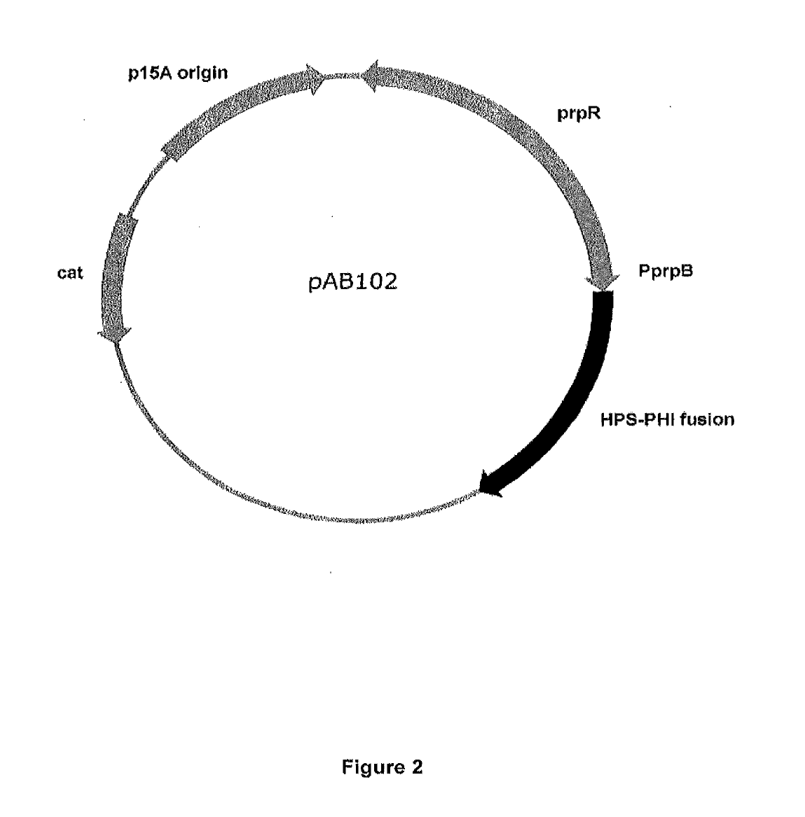

In one embodiment, the microbe is engineered to comprise components of a ribulose monophosphate pathway (RuMP). Preferably, the components of a ribulose monophosphate pathway are derived from a methylotroph. In another embodiment, the microbe can be engineered to express a gene encoding FMR activity under tight, inducible control. In another embodiment, the FMR and RuMP genes are expressed from inducible promoters that can be differentially regulated with different inducer molecules to allow fine tuning of their respective activity levels (FIGS. 1 and 2). The combined activity of FMR and the RuMP pathway allows for the production of ethanol with increased yield and reduced CO₂ enabled by the utilization of reducing power from an auxiliary substrate, as discussed elsewhere herein.

In one embodiment, establishment of the RuMP pathway in an organism requires hexulose phosphate synthase (HPS) and phosphohexulose isomerase (PHI). In some instances, expression of HPS and PHI can (1) reduce inhibitory or toxic effects of formaldehyde on growth, and (2) increase biomass formation through formaldehyde assimilation when carbon (e.g. glucose) is limiting. In one embodiment, the HPS and PHI genes are expressed as individual soluble proteins. In another embodiment, the HPS and PHI genes are expressed as a transcriptional fusion to produce a bi-functional enzyme comprising both HPS and PHI activities (FIG. 2).

In one preferred embodiment, FMR enzyme is used to shunt carbon into the RuMP pathway by reducing formate to formaldehyde coupled with oxidation of a reduced cofactor. The microorganism of the invention is genetically modified to have the expression of FMR tightly regulated to control production of formaldehyde, which is toxic, and the recipient strain contains the engineered RuMP pathway to protect against formaldehyde production. Controlled expression of FMR can be accomplished by first inactivating any native genes encoding FMR that might be expressed at undesirable levels, and subsequently cloning the FMR gene under the control of an inducible promoter (FIG. 1). In one embodiment, the reduced cofactor oxidized by FMR is NADH. In another embodiment, the reduced cofactor oxidized by FMR is NADPH.

An alternate pathway that can be used for reduction of formate to formaldehyde is the tetrahydrofolate (THF) pathway, in which a series of enzymatic steps are used to convert formate, THF, ATP, and NAD(P)H into 5,10-methylene-THF, which spontaneously disassociates to produce formaldehyde and reform THF (Lidstrom 2006, Prokaryotes 2: 618-634). Similar pathways involving other cofactors such as tetrahydromethanopterin (H4MPT) are found in other organisms (Lidstrom 2006, Prokaryotes 2: 618-634), and such pathways may also be employed to reduce formate to formaldehyde.

In one embodiment, the microbe is engineered to express the genes encoding the folate-linked THF pathway for formate reduction to formaldehyde. In another embodiment, the microbe is engineered to express the genes encoding synthesis of the H4MPT cofactor, and the genes encoding the H4MPT-linked pathway for formate reduction to formaldehyde.

Alternate pathways for carbon assimilation that may be employed include (1) the “serine” pathway for formaldehyde assimilation found in “Type H” methylotrophic bacteria (Lidstrom 2006, Prokaryotes 2: 618-634), the xylulose monophosphate (XuMP) pathway for formaldehyde assimilation found in methylotrophic yeasts (Yurimoto et al. 2005 The Chemical Record, 5: 367-375), or the Calvin-Benson-Bassham (CBB) pathway that fixes carbon dioxide and which is found in many autotrophic bacteria. Generally, the RuMP pathway is preferred because it is well characterized, requires heterologous expression of the fewest enzymes in foreign host organisms, and being exergonic, is the most energy efficient. A potential advantage of the serine pathway for formaldehyde assimilation is that fixation of carbon dioxide catalyzed by the pathway allows up to four moles of ethanol to be produced per mole of glucose; however, this requires more reducing power and consumes ATP.

In one embodiment, the microbe is engineered for heterologous expression of the genes necessary for the “serine” pathway. In another embodiment, the microbe is engineered for heterologous expression of the genes necessary for the XuMP pathway. In a preferred embodiment, the microbe engineered to express the XuMP pathway is a yeast.

Reducing Power from Auxiliary Sources.

A microorganism engineered with the genes encoding FMR and the RuMP pathway requires 6 additional moles of NADH, which is equivalent to 12 moles of electrons (FIG. 6), to convert one mole of glucose into three moles of ethanol without carbon dioxide production. Given the required reducing power, the engineered microorganism can thus eliminate up to 100% of the CO₂ produced during fermentation, and produce ethanol with up to a 50% increase in yield when compared to current state-of-the-art fermentation pathways utilized by yeast or other engineered microorganisms (e.g. E. coli or Zymomonas mobilis), which produce two moles of ethanol per mole of glucose. When lower levels of supplemental reducing power are provided, some reduction in CO₂ production may still be realized, and ethanol yields of between 2 and 3 moles ethanol per mole glucose are achieved.

The reducing power required to reduce formate into ethanol can be provided by various different “auxiliary” electron sources, as described elsewhere herein. A common feature of these sources is that they are inexpensive relative to the value of ethanol product, and their use either does not give rise to CO₂, or gives rise to much less CO₂ than current processes. In a preferred embodiment, reducing power is provided to the cell using a combination of auxiliary electron sources.

In one embodiment, an enzyme is used to supply electrons to the cell through the enzymatic oxidation of a non-fermentable reduced chemical substrate, wherein the oxidation reaction does not produce CO₂. For example, phosphite dehydrogenase (PTDH), which catalyzes the largely irreversible oxidation of hydrogen phosphonate (phosphite) to phosphate with reduction of NAD+ to NADH (Relyea and van der Donk, Bioorg Chem, 2005. 33(3): p. 171-89; Vrtis et al., Angew Chem Int Ed Engl, 2002. 41(17): p. 3257-9), is introduced into an organism in order to facilitate oxidation of phosphite for NADH regeneration (FIG. 6). In this way, PTDH can supply reducing power to the cell to drive the reduction of formate to ethanol. PTDH provides an ideal system because the reaction is not directly involved in sugar metabolism or ethanol production. The reaction is exergonic and essentially irreversible, and NADH production can be modulated by varying the concentration of phosphite substrate. In some instances, PTDH can be expressed from a replicating plasmid under control of an inducible promoter. Different concentrations of phosphite can be added to the growth medium to produce varying amounts of intracellular NADH.

In one embodiment, the reducing power is made available to the cell through incubation of the engineered microbe in the presence of reduced chemicals. For example, reduced compounds applicable to the invention include but is not limited to anthrahydroquinone-2,6-disulfonate (AH2QDS) and iron (II) sulfate (FeSO₄), which can be oxidized by the microbe to provide reducing power to the cell without producing CO₂. Other molecules that undergo reduction-oxidation (redox) reactions and have the appropriate midpoint potentials for transfer of electrons to NAD+ can also be used to provide reducing power to the cell and will be known to those skilled in the art armed with the present disclosure. For example, lignin fragments, humic substances, phenazine, and quinones all act as shuttles in metabolism or in microbial fuel cells and may be used for this purpose (Coates et al., Appl Environ Microbiol, 2002. 68(5): p. 2445-52; von Canstein et al., Appl Environ Microbiol, 2008. 74(3): p. 615-23; Jung and Regan, Appl Microbiol Biotechnol, 2007. 77(2): p. 393-402; Zhang et al., Electrochemistry communications, 2008. 10: p. 293-297; Sund et al., Appl Microbiol Biotechnol, 2007. 76(3): p. 561-8).

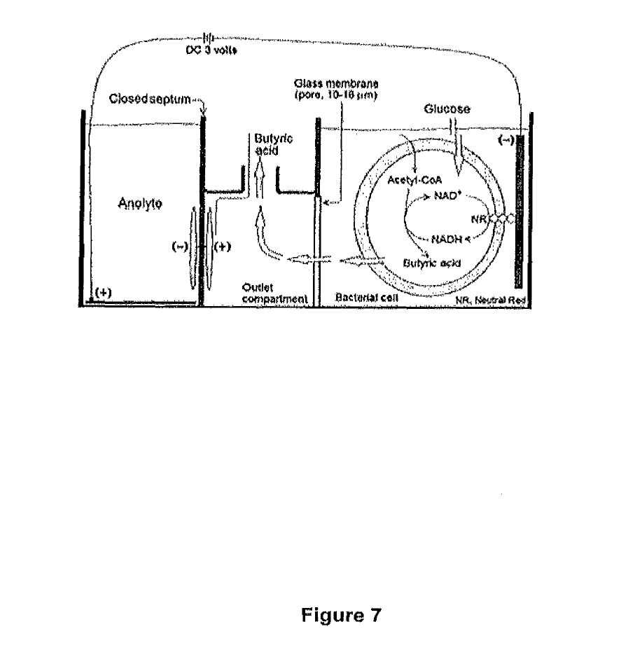

In one embodiment, reducing power to increase the conversion of feedstock to ethanol is provided to the cell from an electric power source via an electrochemical bioreactor (Hwang et al. 2008, Biotechnol Bioprocess Eng 13: 677-682) (FIG. 7). This system has the advantage of neither requiring additional feedstocks to be added to the engineered microbe as external reductant, nor depositing oxidation products in the spent fermentation broth. Addition of an electron transport mediator or electron shuttle to the electrochemical bioreactor is anticipated to be required to facilitate a high rate of electron transport between the cathode and the engineered microbe. The electrochemical bioreactor regenerates the reduced form of the mediator after it is has been oxidized by the cell, thus requiring relatively low “catalytic” concentrations to be used. A variety of molecules that undergo reduction-oxidation (redox) reactions and have the appropriate midpoint potentials can be used as mediators in the electrochemical bioreactor system, and will be known to those skilled in the art armed with the present disclosure. For example, AH2QDS, iron (II) sulfate (FeSO₄), lignin fragments, humic substances, phenazine, and quinones all act as shuttles in metabolism or in microbial fuel cells and may be used as mediators in a electrochemical bioreactor (Coates et al., Appl Environ Microbiol, 2002. 68(5): p. 2445-52; von Canstein et al., Appl Environ Microbiol, 2008. 74(3): p. 615-23; Jung and Regan, Appl Microbiol Biotechnol, 2007. 77(2): p. 393-402; Zhang et al., Electrochemistry communications, 2008. 10: p. 293-297; Sund et al., Appl Microbiol Biotechnol, 2007. 76(3): p. 561-8).

In one embodiment, a simple electrochemical bioreactor based upon the anthraquinone disulfonate (AQDS)/anthrahydroquinone disulfonate (AH2QDS) anthraquinone shuttle system (i.e. mediator) can be used to deliver reducing power to the cell to drive the regeneration of NAD(P)H in the cytoplasm. AQDS enters the E. coli cytoplasm and may interact directly with central metabolism. AQDS has been employed as an analog of lignin fragments. In a preferred embodiment, lignin fragments are a convenient choice of electron shuttle when the microbe is fermenting a cellulosic biomass feedstock, since they are already present in the pretreated biomass feedstock and thus would add little or no cost. In one embodiment, the electron mediator is added to the fermenter with the microbe and fermentation feedstocks. In a preferred embodiment, the electron mediator is produced and secreted into the fermenter by the engineered microbe. In some instances, corn feed stock can be used in the invention.

In one embodiment, the energy contained in the fermentation process waste stream is captured through combustion and use of a boiler and turbogenerator to produce electrical energy that is used to drive the production of additional ethanol by the engineered microorganism. Initial calculations suggest that the combustion of fermentation solids from a cellulosic ethanol plant as forecast by the U.S. Department of Energy (Aden et al., 2002 Lignocellulosic Biomass to Ethanol Process Design and Economics Utilizing Co-Current Dilute Acid Prehydrolysis and Enzymatic Hydrolysis for Corn Stover. National Renewable Energy Laboratory, Golden Colo.) provides enough reducing power to increase the ethanol yield by about 12.5% with no additional cost. In one embodiment, ethanol yield increases are achieved by purchasing additional electricity for delivery to the electrochemical bioreactor. In one embodiment, improved methods for electricity generation are used to reduce the cost of electricity generation and/or use.

### Transhydrogenase

Some enzymes expressed in the engineered microorganism may require NADPH or NADH as reduced cofactor. For example, the FMR enzymes may require NADPH as the reduced cofactor. In one embodiment, reducing power is transferred between the intracellular pools of NADH and NADPH by heterologous expression of the soluble transhydrogenase (STH) from Pseudomonas fluorescens, or a related protein, which catalyzes freely reversible reduction-oxidation reactions between NADH and NADPH (Boonstra 2000, App Env Microbiol, 66: 5161-5166). Armed with the present disclosure, a skilled artisan can use other approaches to ensure that sufficient levels of the reduced cofactors required by the engineered microbe are available for metabolism.

Reducing Power Produced from Enzymatic Lignin Oxidation.

During fermentation of cellulosic biomass by an engineered microorganism containing the FMR and RuMP genes (or one or more of the alterative enzymes and pathways described above), reducing power as NAD(P)H can be produced via enzymatic oxidation of lignin in the feedstock to drive the production of increased ethanol yield and reduced co-production of CO₂. NAD(P)H can be produced by the enzymatic oxidation of hydroxyl and/or carbonyl groups of lignin present in biomass feedstocks. One type of enzyme suitable for this reaction is a NAD-dependent oxidoreductase. Several unique NAD-dependent oxidoreductase enzymes have been reported in the literature to catalyze the oxidation of lignin with the coupled reduction of NAD+ to NADH, including LigD from Sphingomonas paucimobils SYK-6 (Masai et al., Biosci Biotechnol Biochem, 2007. 71(1): p. 1-15; Sato et al., Appl Environ Microbiol, 2009. 75(16): p. 5195-5201), and several enzymes from Pseudomonas species: GGE-DH1 and GGE-DH2 (Pelmont et al., 1985, Biochimie 67:973-986; Pelmont et al. 1989 FEMS Microbiol Lett 57:109-114), DH (Vicuna et al., Appl Environ Microbiol, 1987. 53(11): p. 2605-2609), and DH-I and DH-II (Habu et al., Agric Biol Chem, 1988. 52(12): p. 3073-3079).

Other microorganisms that catabolize lignin are predicted to produce similar oxidoreductase enzymes that can oxidize lignin coupled with NAD(P)+ reduction to NAD(P)H. Such enzymes can be identified for use in the current invention through established experimental approaches for cloning and screening new enzymes for lignin oxidation coupled to NAD+ reduction to NADH. Such enzymes may also be identified through bioinformatic methods that can predict NAD-linked oxidoreductase enzymes with lignin-oxidizing activity based on their amino-acid sequence identity to enzymes known to posses this activity and substrate specificity.

Strategies for lignin oxidation include expression of the cloned enzymes in: (1) the extracellular environment, (2) the periplasmic space, or (3) the cell cytoplasm. The preferred approach will depend of the structure and size of lignin fragments available to the engineered microbe, and their ability to enter either the periplasm or the cytoplasm. A combination of approaches may be employed. One approach of the invention depends in part on the extent of lignin degradation and its resulting properties after the physical and/or chemical pretreatment process that is used prior to fermentation to break apart both cellulose fibers, and potentially, lignin.

In one embodiment, oxidoreductase enzymes expressed in the cytoplasm oxidize lignin fragments that enter the cell and directly reduce the intracellular pool of NAD+ to NADH. In one embodiment, lignin transporters are used to improve the transport of lignin fragments into the cell. Such lignin transporters have been proposed in bacteria that grow on lignin, but are not yet known (Masai et al., Biosci Biotechnol Biochem, 2007. 71(1): p. 1-15). Putative lignin transporters can be identified from such bacteria by screening clone libraries for the ability to produce NADH when incubated with lignin fragments. Screening can be performed using methods known to those skilled in the art. For example, a solid-phase (colony-based) assay, or a spectrophotometer-based assay performed in multi-well plates can be used.

In one embodiment, oxidoreductase enzymes secreted into the periplasmic space oxidize lignin fragments that enter the periplasm. In one embodiment, oxidoreductase enzymes secreted into the extracellular environment oxidize lignin fragments that remain outside the cell. Both secretion approaches are accomplished by including the appropriate secretion tag in the expressed protein sequence using established methods (Mergulhao et al. 2005, Biotechnol Adv 23: 177-202). In one embodiment, various diffusible redox-active mediators having the appropriate redox potential for electron transfer with NAD+/NADH may be used to improve electron transfer to the cell from lignin oxidation catalyzed in either the periplasm or extracellular environment. For example, humic substances, AQDS, phenazine, and quinones all act as shuttles in metabolism or in microbial fuel cells and may be used for this purpose (Coates et al., Appl Environ Microbiol, 2002. 68(5): p. 2445-52; von Canstein et al., Appl Environ Microbiol, 2008. 74(3): p. 615-23; Jung and Regan, Appl Microbiol Biotechnol, 2007. 77(2): p. 393-402; Zhang et al., Electrochemistry communications, 2008. 10: p. 293-297; Sund et al., Appl Microbiol Biotechnol, 2007. 76(3): p. 561-8). In one embodiment, the engineered microbe contains heterologous genes expressing proteins that facilitate electron transfer between the cell and extracellular redox mediators. For example, CymA (from Shewanella oneidensis) enabled E. coli to grow as a dissimilatory iron-reducing bacterium, and enhanced periplasmic electron transfer between the cell and AQDS. Expression of CymA or other related redox-active proteins may thus be used to improve transfer of electrons harvested from lignin oxidation into the cell.

Thus, a microorganism can be engineered to express or to have the desired oxidoreductase enzyme active so that the microorganism is able to oxidize lignin. Such an engineered microorganism is advantageous because the microorganism is able to both reduce CO₂ production and increase a desirable fermentation product by being able to oxidize lignin rather than a desired carbon source as a means to maintain the redox balance. The engineered microorganism can produce more fermentation product because the flow of carbon hitherto destined for CO₂ production is redirected into a pathway to produce additional fermentation product. This is because equimolar CO₂ production is not required if a substrate other than fermentable hexose or pentose sugars is available for oxidation.

In one embodiment, the invention provides a modified microorganism so that the microorganism produces a desired product such as ethanol wherein CO₂ production is reduced compared to the amount of CO₂ produced by an otherwise identical microorganism not modified according to the present invention. In some embodiments, the microorganism is modified to encode a type of oxidoreductase enzyme so that the modified microorganism produces ethanol more efficiently because the microorganism is able to oxidize lignin in order to reduce CO₂ production in the fermentation process.

In another embodiment, the microorganism is modified to encode a more active form of a type of oxidoreductase so that the modified microorganism produces ethanol more efficiently because the microorganism is able to oxidize lignin in order to reduce CO₂ production in the fermentation process.

In yet another embodiment, the microorganism is modified to have a type of oxidoreductase activated so that the modified microorganism produces ethanol more efficiently because the microorganism is able to oxidize lignin in order to reduce CO₂ production in the fermentation process.

### Inactivation of Native Genes.

E. coli can be used as the host organism in order to leverage the powerful genetic tools and knowledge base available for metabolic engineering and heterologous protein expression in this organism. However, analogous approaches could be used to engineer any suitable microorganism to achieve similar results for commercial ethanol production. For example, a strain of yeast or Zymomonas mobilis could be engineered for increased ethanol production and reduced carbon dioxide production during fermentation.

In one embodiment, non-essential enzymes that catalyze reactions detrimental to the production of ethanol by the engineered microbe are inactivated. For example, the engineered microbe may contain one or more advantageous mutations to (1) increase the flow of carbon through the engineered metabolic pathways be eliminating competing reactions, (2) reduce or eliminate production of undesirable metabolic waste products, and (3) prevent futile cycles created between native and engineered metabolic pathways.

The products of mixed acid fermentation, performed naturally by microorganisms such as E. coli, are succinate, lactate, acetate, ethanol, formate, carbon dioxide, and hydrogen gas. In one embodiment, the native E. coli formate hydrogen lyase (FHL) complex is inactivated. This mutation eliminates a competing pathway for formate utilization in which carbon and energy are lost from the cell when formate is oxidized into carbon dioxide and hydrogen gas, respectively. Inactivating the FHL complex allows substantially all of the formate produced by pyruvate formate lyase (PFL) during fermentation to be utilized by the engineered pathway comprising formate reductase (FMR) and the ribulose monophosphate enzymes (RuMP).

In one embodiment, the native E. coli lactate dehydrogenase (LDH) enzyme is inactivated. LDH is responsible for lactate production, which is an undesirable waste product during ethanol fermentation because production of lactate reduces the yield of carbon recovered as the desired ethanol product. In one embodiment, the native E. coli fumarate reductase (FRD) enzyme is inactivated. FRD is responsible for succinate production, which is an undesirable waste product during ethanol fermentation because production of succinate reduces the yield of carbon recovered as the desired ethanol product.

In one embodiment, the native E. coli pathway for oxidation of formaldehyde to formate catalyzed by glutathione-dependent formaldehyde dehydrogenase (GS-FDH) and S-formylglutathione hydrolase (FGH) is inactivated. The GS-FDH and FGH enzymes create a futile cycle that works against the engineered formate reductase (FMR) enzymes, and elimination of this pathway improves the yield of formaldehyde from formate catalyzed by FMR.

An important improvement in the production of ethanol using modified microorganisms can be achieved by operating temperatures at increased levels at which the ethanol is conveniently removed in a vaporized form from the fermentation medium. Thus, the invention includes the use of thermophilic, ethanologenic bacteria as a host cell for use in the fermentation process for producing ethanol.

The invention encompasses expression vectors and methods for the introduction of exogenous DNA into cells with concomitant expression of the exogenous DNA in the cells such as those described, for example, in Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

The invention also encompasses mutating a gene in a microorganism to render the gene ineffective. The term mutagenesis can be associated with at least three distinct modifications of a DNA fragment (i.e., deletion, insertion, and substitution). Deletion corresponds to removal of one or more nucleotides from the DNA fragment of interest; insertion corresponds to addition of same; substitution corresponds to replacement of one or more bases with a same number of bases of different nature.

The invention encompasses vectors comprising the nucleic acid sequences, open reading frames and genes of the invention, as well as host cells containing such vectors.

The term “vector” is used to refer to a nucleic acid molecule into which a nucleic acid sequence can be inserted for introduction into a cell where it can be replicated and/or expressed. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. One of skill in the art would be well equipped to construct a vector through standard recombinant techniques, which are described in Sambrook et al., MOLECULAR CLONING: A LABORATORY MANUAL, volumes 1-3 (3rd ed., Cold Spring Harbor Press, NY 2001), and Ausubel et al. (1997, Current Protocols in Molecular Biology, John Wiley & Sons, New York), both incorporated herein by reference.

The term “expression vector” refers to a vector containing a nucleic acid sequence coding for at least part of a gene product capable of being transcribed. In some cases, RNA molecules may then be translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism.

A vector typically contains a promoter region. A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment. Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment.

It is advantageous to employ a promoter and/or enhancer that effectively directs the expression of the DNA segment in the cell type chosen for expression. Those of skill in the art of molecular biology generally know the use of promoters, enhancers, and cell type combinations for protein expression, for example, see Sambrook et al., MOLECULAR CLONING: A LABORATORY MANUAL, volumes 1-3 (3rd ed., Cold Spring Harbor Press, NY 2001). The promoters employed may be constitutive, inducible, and/or useful under the appropriate conditions to direct high level expression of the introduced DNA segment, such as is advantageous to grow microorganisms to a greater cell density, increased yield of desired products, increased amount of volumetric productivity, removal of unwanted co-metabolites, improved utilizaton of inexpensive carbon and nitrogen sources, and adaptation to fermenter conditions, increased production of a primary metabolite, increased production of a secondary metabolite, increased tolerance to acidic conditions, increased tolerance to basic conditions, increased tolerance to organic solvents, increased tolerance to high salt conditions, increased tolerance to high or low temperatures, etc.

In order to propagate a vector in a host cell, it may contain one or more origins of replication sites (often termed “ori”), which is a specific nucleic acid sequence at which replication is initiated. The origin of replication may optionally be active or non-active at specific temperatures, i.e., temperature sensitive.

In certain embodiments of the invention, the cells contain nucleic acid construct of the present invention, a cell may be identified in vitro by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers.

Another class of reporter genes which confer detectable characteristics on a host cell are those which encode polypeptides, generally enzymes, which render their transformants resistant against toxins. Examples of this class of reporter genes are the neo gene which protects host cells against toxic levels of the antibiotic G418, the gene conferring streptomycin resistance, the gene conferring hygromycin B resistance, a gene encoding dihydrofolate reductase, which confers resistance to methotrexate, the enzyme HPRT, along with many others well known in the art. Chloramphenicol acetyltransferase (CAT) confer resistance to chloramphenicol, and the β-lactamase gene confers ampicillin resistance.

In accordance with the present invention, nucleic acid sequences are transferred into a desired cell (e.g., bacterial cells) using standard methodologies known to those of ordinary skill in the art. In certain embodiments of the present invention, the vector or otherwise construct is introduced into the cell via electroporation. Electroporation involves the exposure of a suspension of cells and DNA to a high-voltage electric discharge. Electroporation works well with bacteria.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Modified Lignins

Disclosed herein are compositions and methods useful in energy applications, with particular applicability to reducing CO₂, i.e. during the fermentation process of producing ethanol. The compositions and methods described herein may involve the use of modified lignins and formulations thereof Lignin is a naturally-occurring cross-linked, polymerized macromolecule comprised of aliphatic and aromatic portions with alcohol functionality interspersed. Lignin polymers incorporate three monolignol monomers, methoxylated to various degrees: p-coumaryl alcohol, coniferyl alcohol, and sinapyl alcohol. These are incorporated into lignin in the form of the phenylpropanoids, p-hydroxyphenyl, guaiacyl, and syringal respectively. The systems and methods disclosed herein describe how naturally-occurring (i.e., native) and unnatural or modified lignin may be modified through functionalization of the resident alcohol moieties to alter the properties of the polymer. Such a functionalized lignin may be termed a “modified lignin.” The word “lignin”, as used herein is intended to include natural and non-natural lignins which possess a plurality of lignin monomers and is intended to embrace lignin, kraft lignin, lignin isolated from bagasse and pulp, oxidized lignin, alkylated lignin, demethoxylated lignin, lignin oligomers, and the like.

Lignin and oxidized lignin are waste products from the paper industry. Oxidized lignin is characterized by a plurality of hydroxyl groups which can be conveniently reacted. Oxidized lignin is described, for example, in U.S. Pat. No. 4,790,382 and is characterized by a plurality of hydroxyl groups which can be conveniently reacted. Similarly, kraft lignins, such as indulins, including Indulin AT, can be used. For example, the hydroxyl groups can be reacted with succinic anhydride and similar compounds to form a carboxylic acid-substituted lignin, by a ring opening reaction.

Adding a reactive agent such as succinic anhydride or alkylated succinic anhydride to a native lignin may produce a modified lignin of the invention. Alkylated succinic anhydride is commonly used in the paper industry as a sizing agent. The alkyl additions are long chain hydrocarbons typically containing 16-18 carbon atoms. However, alkylated succinic acids having alkyl side chains having more than 1 carbon atom, such as 1 to 30 carbon atoms can be used as well. Such alkyl groups are defined herein to include straight chain, branched chain or cyclized alkyls as well as saturated and unsaturated alkyls. Addition of an anhydride, such as a succinic anhydride or alkylated succinic anhydride, to the resident alcohol groups result in new ester linkages and the formation of carboxylic acids via a ring opening mechanism. Addition of anhydride to the resident alcohol groups result in new ester linkages and/or the formation of carboxylic acids via a ring opening mechanism. With the newly added carboxylic acid functionality, the lignin becomes more water soluble.

Hydroxyl group can be reacted with a dicarboxylic acid, such as maleic acid, or activated esters or anhydrides thereof to form a carboxylic acid substituted lignin. For example, the anhydride derived from many acids can be utilized, such as adipic acid, or the functionality can be derived from natural compounds such as a polysaccharide that contains carboxylic acid groups. Non-limiting examples include pectin or alginate, and the like, and synthesized polymers such as polyacrylic or methacrylic acid homo or co-polymers. Further, activated esters can be used in place of the anhydride. Other examples will be apparent to those of ordinary skill in the art. The degree of functionalization (i.e., the percentage of hydroxyl groups that are reacted to present an ionic moiety) can be between 20% and 80%, preferably between 50% and 80% and any and all whole or partial increments there between.

In other embodiments, lignin (oxidized or native) may be treated by chemically reacting it with reagents to tune the hydrophilicity to present alcohol groups. Examples of such reagents include hydrophilic molecules, or hydrophilic polymers, such as poly(ethylene glycol) (PEG) or polypropylene glycol) (PPO) and combinations thereof. In a preferred embodiment, the hydrophilic polymer can have a molecular weight between 700 and 2500 g/mol. Addition of PEG or PPO (with or without acidification) can be useful in stabilization of the product in salt solutions, particularly divalent cation salts. In this embodiment, the amount of polymer to lignin is preferably added in an amount between 25% and 75%.

### Fermentation

The fermentation process is generally considered to comprise four main categories: pretreatment, hydrolysis of pretreated material, fermentation, and optionally recovery of the fermentation product, such as ethanol. The fermentation process allows for the production of a fermentation product from a biomass, such as a lignocellulosic material. The present invention is an improvement in existing fermentation processes because the invention relates to the discovery that equimolar CO₂ production is not required if a substrate other than fermentable hexose and pentose sugars is available for oxidization.

In some instances, the invention includes both reducing CO₂ production and increasing a desirable fermentation product by oxidizing lignin as a means to maintain the redox balance. Oxidation of lignin allows for the microorganism to maintain the redox balance without the requirement of oxidizing the desired carbon source such as glucose for the production of a product such as ethanol. For example, using lignin as a source of electrons for the reduction of pyruvate to ethanol increases the yield of ethanol produced from the fermentation of biomass by directing the flow of carbon atoms previously utilized for carbon dioxide production into a biosynthetic pathway to produce additional ethanol. Thus the invention is applicable to at least pretreatment, hydrolysis of pretreated material, and fermentation as these processes can be effected by oxidation of lignin.

Typically, the pre-treatment step is carried out to separate and/or release cellulose, hemicellulose, and lignin. The lignocellulosic material may, during the pre-treatment, be present in an amount between 10-80 wt. %, preferably between 20-50 wt. %. The goal is to break down the lignin seal and disrupt the crystalline structure of the lignocellulosic material. The structure of the lignocellulosic material is altered and especially polymeric constituents are made more accessible to enzyme hydrolysis in later process steps where carbohydrate polymers (i.e., cellulose and hemicellulose) are converted into fermentable hexose and pentose sugars. Pre-treatment may be carried out in any suitable way to separate and/or release cellulose, hemicellulose and/or lignin. Examples of suitable pre-treatment methods are described by Schell et al. (2003) Appl. Biochem and Biotechn. Vol. 105-108, p. 69-85, and Mosier et al. Bioresource Technology 96 (2005) 673-686, which are hereby incorporated by reference. In another embodiment, the lignocellulosic material is treated chemically and/or mechanically.

Chemical treatment and mechanical treatment (otherwise referred to as physical treatment) can be used alone or in combination with subsequent or simultaneous enzymatic steps to promote the separation and/or release of cellulose, hemicellulose and/or lignin from lignocellulosic material. Chemical treatment includes any chemical treatment process which can be used to promote the separation and/or release of cellulose, hemicellulose and/or lignin from lignocellulosic material. Non-limiting examples of suitable chemical treatment processes include, acid and base treatment, dilute acid, lime and ammonia pretreatment, wet oxidation, and solvent treatment.

Cellulose solvent treatment has been shown to convert 90% of cellulose to glucose. Also, enzyme hydrolysis can be greatly enhanced when the biomass structure is disrupted. Alkaline H₂O₂, ozone, organosolv (uses Lewis acids, FeCl₃, (Al)₂SO₄ in aqueous alcohols), glycerol, dioxane, phenol, or ethylene glycol are among solvents known to disrupt cellulose structure and promote hydrolysis.

Wet oxidation techniques involve the use of oxidizing agents, such as sulfite based oxidizing agents and the like. Examples of solvent treatments include treatment with DMSO (dimethyl sulfoxide) and the like. Chemical treatment processes are generally carried out for about 5 to about 10 minutes, but may be carried out for shorter or longer periods of time.

Mechanical treatment includes any mechanical or physical treatment process which can be used to promote the separation and/or release of cellulose, hemicellulose and/or lignin from lignocellulosic material. Mechanical treatment includes comminution, which encompasses mechanical reduction in biomass particulate size, steam explosion and hydrothermolysis. Comminution includes dry and wet and vibratory ball milling. Preferably, a mechanical treatment process involves a process which uses high pressure and/or high temperature (steam explosion).

As discussed elsewhere herein, lignocellulosic material is pre-treated to separate and/or release cellulose, hemicellulose and/or lignin. These carbohydrate polymers can be converted into monomeric sugars. For example, cellulose can be hydrolyzed to form glucose either chemically or enzymatically using a cellulase.

Hemicellulose polymers can be broken down by hemicellulases or acid hydrolysis to release its five and six carbon sugar components. The six carbon sugars (hexoses), such as glucose, galactose and mannose, can readily be fermented to, e.g., ethanol, acetone, butanol, glycerol, citric acid and fumaric acid, by a suitable fermenting organism.

The fermentation of microorganisms for the production of natural products is a widely known application of biocatalysis. The present invention offers an improvement to existing fermentation processes in that CO₂ production can be reduced and an increase yield of product produced can be accomplished. Industrial microorganisms effect the multistep conversion of renewable feedstocks to high value chemical products in a single reactor and in so doing catalyze a multi-billion dollar industry. Fermentation products range from fine and commodity chemicals such as ethanol, lactic acid, amino acids and vitamins, to high value small molecule pharmaceuticals, protein pharmaceuticals, and industrial enzymes.

Success in bringing these products to market and success in competing in the market depends partly on continuous improvement of the whole cell biocatalysts. Improvements include the ability to grow microorganisms to a greater cell density, increased yield of desired products, increased amount of volumetric productivity, removal of unwanted co-metabolites, improved utilizaton of inexpensive carbon and nitrogen sources, and adaptation to fermenter conditions, increased production of a primary metabolite, increased production of a secondary metabolite, increased tolerance to acidic conditions, increased tolerance to basic conditions, increased tolerance to organic solvents, increased tolerance to high salt conditions and increased tolerance to high or low temperatures. Shortcomings in any of these areas can result in high manufacturing costs, inability to capture or maintain market share, and failure of bringing promising products to market.

Fermentation includes, without limitation, fermentation methods or processes used to produce any fermentation product, including alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. In a preferred embodiment the fermentation step is an alcohol fermentation process. More preferably, the fermentation process is anaerobic.

The term “fermenting organism” refers to any organism, including bacterial and fungal organisms, suitable for producing a desired fermentation product. Especially suitable fermenting organisms according to the invention are able to ferment, i.e., convert, sugars, such as xylose and/or glucose, directly or indirectly into the desired fermentation product. Examples of fermenting organisms include fungal organisms, such as yeast.

In one embodiment of the present invention, the host cell having the above mentioned attributes is also ethanologenic. Accordingly, the invention provides methods for producing ethanol using such host cells (or extracts/enzymes derived therefrom). In addition, the host cells can be used in degrading or depolymerizing a complex saccharide into a monosaccharide. Subsequently, the cell can catabolize the simpler sugar into ethanol by fermentation. This process of concurrent complex saccharide depolymerization into smaller sugar residues followed by fermentation is referred to as simultaneous saccharification and fermentation (SSF).

In another embodiment, the host cell is thermophilic. A thermophilic microorganism has the characteristics of being able to ferment sugars aerobically, as wells as being active in fermentation at 70° C. or above. In some instance, a thermophilic microorganism has the characteristics of being able to ferment sugars anaerobically, as wells as being active in anaerobic fermentation at 70° C. or above. In some instances, a thermophilic microorganism has the characteristics of being able to ferment sugars aerobically and anaerobically, as wells as being active in anaerobic fermentation at 70° C. or above.

In other instances, the anaerobic fermentation can be carried out with continuing removal of ethanol at 70° C. In some instances, the fermentative activity of the microorganism is maintained by withdrawing a proportion of the anaerobic fermentation medium on a continuing basis, preferably with removal of ethanol, and allowing the microorganism therein to multiply aerobically, using residual sugars or metabolites thereof present in the medium, before being returned to the anaerobic fermentation.

The methods and compositions of the present invention can be adapted to conventional fermentation bioreactors (e.g., batch, fed-batch, cell recycle, and continuous fermentation) to improve the fermentation process wherein CO₂ production is reduced. As such, the methods disclosed herein in turn increases the profitability of current fermentation processes and can facilitate the development of new products.

The skilled artisan will also recognize, based on the disclosure set forth herein, that a multitude of organisms, techniques, and metabolic pathways are available for use in the present invention, and that various fermentation products can be obtained as desired according to the present invention.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

### Controlled Expression of Formate Reductase in E. coli

Tani et al (Agric Biol Chem, 1978, 42: 63-68; Agric Biol Chem, 1974, 38: 2057-2058) showed that purified enzymes from Escherichia coli strain B could reduce the sodium salts of different organic acids (e.g. formate, glycolate, acetate, etc.) to their respective aldehydes (e.g. formaldehyde, glycoaldehyde, acetaldehyde, etc.). Of three purified enzymes examined by Tani et al (1978), only the “A” isozyme was shown to reduce formate to formaldehyde. Collectively, this group of enzymes was originally termed glycoaldehyde dehydrogenase; however, their novel reductase activity led the authors to propose the name glycolate reductase as being more appropriate (Morita et al, Agric Biol Chem, 1979, 43: 185-186). Morita et al (Agric Biol Chem, 1979, 43: 185-186) subsequently showed that glycolate reductase activity is relatively widespread among microorganisms, being found for example in: Pseudomonas, Agrobacterium, Escherichia, Flavobacterium, Micrococcus, Staphylococcus, Bacillus, and others. Without wishing to be bound by any particular theory, it is believed that some of these glycolate reductase enzymes are able to reduce formate to formaldehyde.

### Identification of Genes Encoding Formate Reductase Enzymes.

Experiments were designed to identify genes encoding organic acid reductase or aldehyde dehydrogenase enzymes that can reduce formate to formaldehyde. For example, bioinformatics-based approaches (e.g. DNA and/or amino-acid sequence analysis) known to those skilled in the art are employed to identify genes with similarity to other known or predicted acid reductase or aldehyde dehydrogenase enzymes. For example, the genome of E. coli strain B121(DE3) contains many genes annotated as aldehyde dehydrogenases that may have FMR activity; some of these genes include GI:253977584 (SEQ ID NO: 1), GI:253323393 (SEQ ID NO: 2), GI:253324554 (SEQ ID NO: 3), GI:253324742 (SEQ ID NO: 4), GI:253325744 (SEQ ID NO: 5), GI:253323698 (SEQ ID NO: 6), GI:253979572 (SEQ ID NO: 7), GI:253977614 (SEQ ID NO: 8), GI:253324665 (SEQ ID NO: 9), and GI:253323522 (SEQ ID NO: 10).

In another approach, acid reductase or aldehyde dehydrogenase enzymes may be identified by empirical testing in the laboratory using, for example, reporter genes and/or activity screening assays, both of which are known to those skilled in the art. For example, a collection of genes can be tested for FMR activity by growing E. coli strains containing each gene under conditions in which the target gene is expressed.

Cell-free extracts are prepared from the bacterial cells, and each extract is tested for FMR activity in an assay in order to identify those genes encoding enzymes with FMR activity. For example, crude cell extracts are produced by collecting the cells from liquid growth medium by centrifugation, washing cells in saline, and releasing cell contents through either the addition of a membrane-disruptive agent, such as lysozyme and/or detergent, or through mechanical methods such as sonication. For example, the reductase activity of each crude extract is assayed in vitro, essentially as described by Tani et al (Agric Biol Chem, 1974, 38: 2057-2058). The assay mixture contains 10 micromoles of the substrate as a sodium salt (e.g. sodium formate, or sodium glycolate), 0.2 micromoles of reduced cofactor (e.g. NADPH or NADH), 1.0 micromoles dimercaprol, 50 micromoles potassium phosphate buffer pH 7.1, and 1.5 mg of the protein being tested in a total volume of 2.82 milliliters. The reaction is monitored in a spectrophotometer at 340 nanometers to measure conversion of NADPH to NADP+ during reduction of the substrate catalyzed by the reductase enzyme.

Genes having FMR activity can be identified using an engineered bacterial strain that expresses a reporter gene in response to formaldehyde production. For example, the frmAB operon in E. coli is induced in response to formaldehyde (Gonzalez 2006, J Biol Chem, 281: 14514-14522). A reporter strain can be constructed by creating a transcriptional fusion between the promoter for the frmAB genes and the E. coli lacZ gene, which encodes beta-galactosidase, or another suitable reporter gene such as green fluorescent protein (GFP). When the frm-lacZ reporter strain expresses a gene encoding FMR activity, and formate is present in the cell (e.g. produced naturally from native metabolic pathways, or made available through addition of formate to the medium), production of formaldehyde by the FMR enzyme will induce expression of lacZ. LacZ activity can be quantified in cell-free extracts in an enzyme assay with the colorimetric substrate 5-bromo-4-chloro-3-indolyl-beta-D-galactopyranoside (X-gal). Alternatively, LacZ activity can be detected in a solid-phase assay by cultivating the bacteria on solidified growth media in the presence of X-gal. If the test strain expresses FMR activity, blue colonies will be formed and the degree of blue color will correlate with the level of expressed FMR activity. In contrast, a strain lacking FMR activity will express low levels of lacZ and form lighter blue or white colonies in the presence of X-gal.

### Cloning of Formate Reductase Genes.

Enzymes having formate reductase activity are cloned into standard expression vectors using previously described methods (see generally, Sambrook et al., 2001, Molecular Cloning: A Laboratory Manual, Third Edition, Cold Spring Harbor Laboratory, New York; Ausubel et al., 1997, Current Protocols in Molecular Biology, John Wiley & Sons, New York, and in Gerhardt et al., eds., 1994, Methods for General and Molecular Bacteriology, American Society for Microbiology, Washington, D.C.). In one embodiment of the invention, a DNA fragment containing the E. coli tac promoter sequence (a fusion of the trp and lac promoters) and the constitutively expressed lacI^{Q} repressor gene are amplified by PCR and cloned into a derivative of the pBR322 plasmid vector which contains the bla gene conferring resistance to ampicillin, and the ColE1 origin of replication. Another DNA fragment containing the gene encoding formate reductase (FMR) enzyme is then amplified by PCR and cloned into the plasmid downstream of the tac promoter (FIG. 1). In E. coli, the tac promoter allows for inducible, differential expression of downstream genes in response to the concentration of isopropyl beta-D-1-thiogalactopyranoside (IPTG) inducer molecule added to the growth media. Other inducible promoter sequences can also be employed for differential expression and based on the disclosure set forth herein, would be understood by those skilled in the art. In another example, constitutive promoters could also be employed.

Tight control over the expression of FMR genes is useful because production of formaldehyde from formate in the cell can be toxic. Approaches to limiting FMR enzyme expression include, for example: (1) use of tightly controlled promoter systems such as the arabinose-inducible bad promoter (Guzman et al, 1995, J Bacteriol, 177: 4121-4130), (2) addition of glucose to repress transcription from certain promoters (e.g. Ptac or Pbad) via catabolite repression, (3) use of low copy-number plasmids to reduce the number of copies of FMR genes, or (4) integration of a single copy of the cloned FMR gene into the chromosome. Importantly, formaldehyde toxicity is reduced when the cell contains a native or engineered pathway for assimilation of carbon from formaldehyde, which is a primary feature of the present invention. Engineering E. coli to have such a pathway is discussed in more detail elsewhere herein.

### Example 2

### Expression of the Ribulose Monophosphate Pathway in E. coli

Collectively, the ribulose monophosphate (RuMP) pathway converts formaldehyde into glyceraldehyde-3-phosphate (G3P) (FIG. 5). In order for this pathway to function in E. coli, two key RuMP pathway enzymes must be cloned and expressed: hexulose phosphate synthase (HPS; reaction 1); and phosphohexulose isomerase (PHI; reaction 2). The remaining reactions are catalyzed by native E. coli enzymes. Ribulose 5-P cofactor is regenerated by multiple sugar-rearrangements catalyzed by pentose phosphate and glycolysis pathway enzymes (reactions 3-5). Dihydroxy-acetone-phosphate is converted into G3P by triosephosphate isomerase (reaction 6). G3P is a glycolysis intermediate that can be converted into pyruvate, and ultimately, ethanol.

Several studies have shown that RuMP genes can be heterologously expressed in other organisms in order to assimilate C1 carbon or detoxify formaldehyde (Mitsui et al. J Bacteriol, 2000, 182(4): p. 944-8.; Yurimoto et al. FEMS Microbiol Lett, 2002. 214(2): p. 189-93.; Yasueda et al. J Bacteriol, 1999. 181(23): p. 7154-60.; Orita et al. J Bacteriol, 2006. 188(13): p. 4698-704.). In one example, Orita et al (2007, Appl Microbiol Biotechnol, 76: 439-445) showed that the HPS and PHI enzymes from the methylotrophic bacterium Mycobacterium gastri could be combined in a translational fusion to produce a single bi-functional polypeptide that was active in E. coli. The bi-functional enzyme conferred higher levels of formaldehyde resistance to E. coli when cultivated in the presence of formaldehyde. Those skilled in the art will recognize that it is similarly possible to express the HPS and PHI enzymes individually, or utilize HPS or PHI genes from other organisms. Genes having demonstrated or predicted HPS and PHI activity are found in diverse microorganisms from Bacteria and Archaea, including both methylotrophic and non-methylotrophic organisms (Mitsui et al. J Bacteriol, 2000, 182: p. 944-8; Vorholt et al. J Bacteriol, 2000, 182: p. 6645-6650).

A translational fusion of the HPS and PHI enzymes from Mycobacterium gastri is cloned and expressed in E. coli, essentially as described by Orita et al (2007, Appl Microbiol Biotechnol, 76: 439-445). Optionally, protein expression is improved by substituting amino-acid codons in the target gene that are rarely used in E. coli with ones that are commonly used by the bacterium. Gene cloning is performed using standard expression vectors and previously described methods (see generally, Sambrook et al., 2001, Molecular Cloning: A Laboratory Manual, Third Edition, Cold Spring Harbor Laboratory, New York; Ausubel et al., 1997, Current Protocols in Molecular Biology, John Wiley & Sons, New York, and in Gerhardt et al., eds., 1994, Methods for General and Molecular Bacteriology, American Society for Microbiology, Washington, D.C.). In one embodiment of the invention, a DNA fragment containing the E. coli prpB promoter sequence and its corresponding prpR repressor gene (Lee and Keasling, 2005, App Env Microbiol 71: 6856-6682) are amplified by PCR and cloned into the pBAD33 plasmid vector (Guzman et al, 1995, J Bacteriol, 177: 4121-4130), which contains the cat gene conferring resistance to chloramphenicol, and the p15A origin of replication. The prpB promoter and prpR genes are cloned into pBAD33 to replace the existing araBAD promoter and araC genes. Another DNA fragment containing the translational fusion of HPS and PHI enzymes is then amplified by PCR and cloned into the plasmid downstream of the prpB promoter (FIG. 2). The HPS-PHI fusion is constructed using standard molecular biology techniques. A representative fusion of HPS and PHI is set forth in SEQ ID NO: 11. In E. coli, the prpB promoter allows for inducible, differential expression of downstream genes in response to the concentration of propionate inducer molecule added to the growth media (Lee and Keasling, 2005, App Env Microbiol 71: 6856-6682). Other inducible promoter sequences can also be employed for differential expression and based on the disclosure set forth herein, would be understood by those skilled in the art. In another example, constitutive promoters could also be employed.

The activity of HPS and PHI, both independently and in concert, is assayed in vitro using cell-free crude extracts and reaction mixtures prepared essentially as described by Orita et al (2007, Appl Microbiol Biotechnol, 76: 439-445).

The in vivo activity of the cloned HPS and PHI enzymes is demonstrated by showing that an E coli strain expressing both enzymes is resistant to supplementing the growth medium with 1 mM formaldehyde, which is known to inhibit growth of the parent E. coli strain. When carbon (from glucose) is limiting and formaldehyde is added to the growth medium, HPS and PHI activity can increase biomass formation through formaldehyde assimilation.

### Example 3

### E. coli Strain Constructions

The products of mixed acid fermentation, performed naturally by microorganisms such as E. coli, are succinate, lactate, acetate, ethanol, formate, carbon dioxide, and hydrogen gas. The host E. coli strain contains several advantageous mutations. First, the native E. coli formate hydrogen lyase (FHL) complex is inactivated. This mutation eliminates a competing pathway for formate utilization in which carbon and energy are lost from the cell when formate is oxidized into carbon dioxide and hydrogen gas, respectively. Inactivating the FHL complex allows substantially all of the formate produced by pyruvate formate lyase (PFL) during fermentation to be utilized by the engineered pathway comprising formate reductase (FMR) and the ribulose monophosphate enzymes (RuMP). Second, the native lactate dehydrogenase (LDH) and fumarate reductase (FRD) enzymes are inactivated. LDH and FRD are responsible for lactate and succinate production, respectively, which are undesirable fermentation waste products because their production reduces the yield of carbon recovered as the desired ethanol product. Third, the native pathway for oxidation of formaldehyde to formate by glutathione-dependent formaldehyde dehydrogenase (GS-FDH) and S-formylglutathione hydrolase (FGH) is inactivated. The GS-FDH and FGH enzymes create a futile cycle that works against the engineered formate reductase (FMR) enzymes, and elimination of this pathway improves the yield of formaldehyde from formate catalyzed by FMR.

Inactivation of chromosomal genes is performed essentially by the method of Datsenko and Wanner (2000, PNAS, 97: 6640-6645), using a two-step process that creates a precise stable deletion of the target gene. E. coli derivatives in which the first step of the inactivation process has been performed for most non-essential single genes are available as part of the “Keio” Collection (Baba et al. 2006, Molecular Systems Biology, pp. 1-11) through the Coli Genetic Stock Center (CGSC) at Yale University. Strains in the Keio collection contain a kanamycin (Kan) resistance cassette replacing all but a few amino-acids of each target gene. Mutant E. coli strains are obtained from the CGSC in which each of the following genes has been inactivated with a Kan cassette: (1) lactate dehydrogenase (ldhA::Kan; JW1375-1; CGSC #9216), which is required for lactate production, (2) fumarate reductase (frdA::Kan; JW4115-1; CGSC #10964), which is required for succinate production, (3) formate dehydrogenase (fdhF::Kan; JW4040-2; CGSC #10908), which is a subunit of the formate hydrogen lyase complex required for production of carbon dioxide and hydrogen gas from formate, and (4) glutathione-dependent formaldehyde dehydrogenase (GS-FDH) (frmA::Kan; JW0347-1; CGSC #8536), which is required for oxidation of formaldehyde to formate.

The mutations in the Keio collection are constructed in E. coli strain BW25113 (genotype: F-, Δ(araD-araB)567, ΔlacZ4787(::rrnB-3), λ-, rph-1, Δ(rhaD-rhaB)568, hsdR514) (Baba et al. 2006, Molecular Systems Biology, pp. 1-11), which is compatible with the present invention and provides two useful features for heterologous expression of FMR and RuMP enzymes when these enzymes are produced from the plasmid expression constructs described in Experimental Examples 1 and 2. First, BW25113 does not produce lactose MFS transporter (encoded by lacY), which allows for homogeneous expression of FMR proteins among a population of cells when IPTG is used to induce transcription from the tac promoter, which is a useful strategy for metabolic engineering (Jensen et al., 1993, Eur J Biochem 211: 181-191; Khlebnikov and Keasling 2002, 18: 672-674). Second, BW25113 is prp+, which is required for induction of the prpB promoter with propionate (Lee and Keasling, 2005, App Env Microbiol 71: 6856-6682). Those skilled in the art will recognize based on the present disclosure that any E. coli strain with these alleles can be used for the desired homogeneous expression of heterologous proteins from the plasmids described above, and that use of other expression constructs (e.g. different inducible promoters) may require the use of other corresponding alleles for optimal control of protein expression.

The Kan cassette that replaces each target gene in the method of Datsenko and Wanner (2000, PNAS, 97: 6640-6645) is flanked by FLP recombinase target (FRT) sites, which are substrates for site-specific DNA recombination catalyzed by FLP recombinase. FLP-catalyzed recombination excises the non-replicating Kan resistance element from the target gene, causing the strain to become Kan sensitive and leaving behind a “scar” sequence containing one FRT site. Eliminating the Kan cassette from strain JW4040-2 (fdhF::Kan) is accomplished essentially as described (Datsenko and Wanner, 2000, PNAS, 97: 6640-6645) by transforming the strain with plasmid pCP20 (Cherepanov and Wackernagel 1995, Gene 158: 9-14). Plasmid pCP20 expresses FLP recombinase from a temperature-inducible promoter, contains a temperature-sensitive origin of replication, and contains genes conferring both ampicillin (Amp) and chloramphenicol (Cam) resistance. Stable transformants of JW4040-2 containing pCP20 are obtained at 30 C on solid media containing Kan and Amp or Cam. Excision of the Kan cassette and simultaneous loss of non-replicating pCP20 plasmid is subsequently accomplished by cultivating the strain at 37 C in the absence of all antibiotics. Excision of the Kan cassette is confirmed by testing the strain for sensitivity to Kan, and by testing for the correct DNA junction sequence at the site of the mutation (e.g. by PCR or DNA sequencing). Loss of pCP20 is confirmed by testing the derivative strain for sensitivity to Amp and Cam. The produced Kan-sensitive strain, AB301, contains a deletion of the fdhF gene and is unable to produce carbon-dioxide and hydrogen gas from formate via the FHL complex.

After the Kan cassette has been eliminated to produce strain AB301 (fdhF), successive bacteriophage P1 transductions are performed to introduce other mutations (i.e. ldhA, frdA, and frmA) into the AB301 strain background. Bacteriophage P1kc is obtained from the ATCC (#25404-B1). P1kc transductions are performed by standard protocols (Miller, J. H. 1992. A short course in bacterial genetics. Cold Spring Harbor Laboratory, Cold Spring Harbor, N.Y). For example, a P1kc lysate prepared by growing the bacteriophage on donor strain JW1375-1 (ldhA::Kan) is used to infect cells of AB301, and the surviving cells are cultivated on solid media containing Kan to select for transductants containing the ldhA::Kan mutation. A Kan-resistant transductant is saved as strain AB302K (fdhF ldhA::Kan). Using essentially the same protocol as above, plasmid pCP20 is transformed into AB302K to enable FLP-catalyzed excision of the Kan cassette and produce a Kan-sensitive strain, AB302 (fdhF ldhA), which is unable to produce lactate as a fermentation product.

The P1kc transduction process is repeated to produce a Kan-resistant derivative of AB302 containing the frdA::Kan mutation, known as AB303K (fdhF ldhA frdA::Kan). FLP-catalyzed excision of the Kan cassette from AB303K is used to produce the Kan-sensitive strain AB303 (fdhF ldhA frdA) , which is unable to produce succinate as a fermentation product.

The P1kc transduction process is repeated to produce a Kan-resistant derivative of AB303 containing the frmA::Kan mutation, known as AB304K (fdhF ldhA frdA frmA::Kan). FLP-catalyzed excision of the Kan cassette from AB304K is used to produce the Kan-sensitive strain AB304 (fdhF ldhA frdA frmA), which is unable to oxidize formaldehyde to formate.

### Example 4

### Increased Ethanol Yield from E. coli through Oxidation of Phosphite Reduced Anthraquinone

### Phosphite Dehydrogenase (PTDH)

Reducing power to drive the production of ethanol from formate can be supplied to the cell by enzymatic oxidation of a non-fermentable chemical substrate whose oxidation does not produce CO₂. For example, the enzyme PTDH catalyzes the largely irreversible oxidation of hydrogen phosphonate (phosphite) to phosphate with reduction of NAD+ to NADH (Relyea and van der Donk, Bioorg Chem, 2005. 33(3): p. 171-89; Vrtis et al., Angew Chem Int Ed Engl, 2002. 41(17): p. 3257-9), and this enzymatic reaction can be used to supply reducing power to the cell through direct recycling of the intracellular NADH pool.

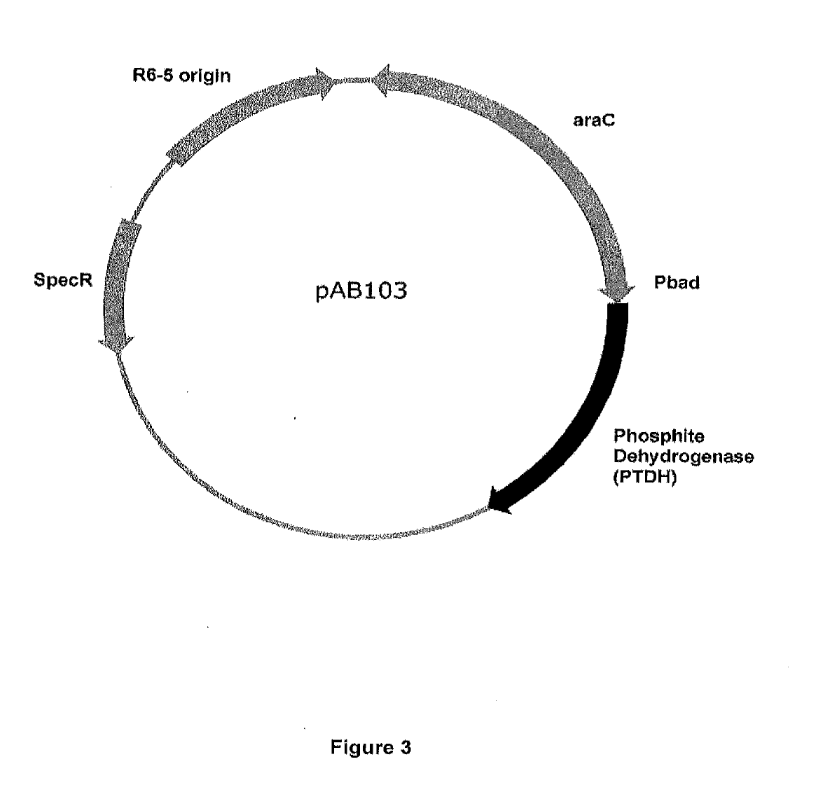

Plasmid pAB 103 (FIG. 3) is constructed in order to express PTDH from a vector that is compatible with the pAB 101 and pAB 102 plasmids described above for expression of the FMR and RuMP genes, respectively (see FIGS. 1 and 2, and Examples 1 and 2). For example, plasmid pSC101 is compatible because it contains the R6-5 origin of replication and contains a gene encoding spectinomycin-resistance. Using standard molecular biology techniques (see generally, Sambrook et al., 2001, Molecular Cloning: A Laboratory Manual, Third Edition, Cold Spring Harbor Laboratory, New York; Ausubel et al., 1997, Current Protocols in Molecular Biology, John Wiley & Sons, New York, and in Gerhardt et al., eds., 1994, Methods for General and Molecular Bacteriology, American Society for Microbiology, Washington, D.C.), a derivative of pSC101 is constructed that contains an arabinose-inducible Pbad promoter and the araC gene encoding its repressor/activator protein, (Guzman et al, 1995, J Bacteriol, 177: 4121-4130). The PTDH gene is subsequently cloned downstream of the arabinose-inducible promoter, which allows induction of PTDH expression in response to addition of arabinose to the growth medium.

Cultivation of a cell containing plasmid pAB 103 allows varying amounts of reducing power to be produced in the cell by adding different concentrations of phosphite to the growth medium. Successful operation of the PTDH system is confirmed by demonstrating increased ratio of ethanol to acetate production during anaerobic growth (using the native E. coli ethanol pathway) (Berrios-Rivera et al., Metab Eng, 2002. 4(3): p. 230-7; Berrios-Rivera et al., Metab Eng, 2002. 4(3): p. 217-29)

or increased activity of a NADH-specific reporter enzyme (Kim et al., Curr Microbiol, 2009. 58(2): p. 159-63) in response to phosphite addition to the cultivation medium. Ethanol and acetate production are determined as described below.

### Reduced Anthraquinones

An alternative approach to provide supplemental reducing power to the cell is to add reduced molecules, such as anthrahydroquinone-2,6-disulfonate (AH2QDS), directly to the growth medium. Oxidation of AH2QDS to anthraquinone-2,6-disulfonate (AQDS) by the bacterium can make additional reducing power available to cytoplasmic metabolic reactions inside the cell (Hatch and Finneran, Curr Microbiol, 2008. 56(3): p. 268-73). Successful regeneration of NADH inside the cell due to AH2QDS oxidation is confirmed by demonstrating an increased ratio of ethanol to acetate production during anaerobic growth (using the native E. coli ethanol pathway) (Berrios-Rivera et al., Metab Eng, 2002. 4(3): p. 230-7; Berrios-Rivera et al., Metab Eng, 2002. 4(3): p. 217-29), or increased activity of a NADH-specific reporter enzyme (Kim et al., Curr Microbiol, 2009. 58(2): p. 159-63) in response to AH2QDS addition to the cultivation medium. Ethanol and acetate production are determined as described below.

Other molecules that undergo reduction-oxidation (redox) reactions and have the appropriate midpoint potentials can also be used and will be known to those skilled in the art based on the present disclosure. For example, humic substances, AQDS, phenazine, and quinones all act as shuttles in metabolism or in microbial fuel cells and may be used for this purpose (Coates et al., Appl Environ Microbiol, 2002. 68(5): p. 2445-52; von Canstein et al., Appl Environ Microbiol, 2008. 74(3): p. 615-23; Jung and Regan, Appl Microbiol Biotechnol, 2007. 77(2): p. 393-402; Zhang et al., Electrochemistry communications, 2008. 10: p. 293-297; Sund et al., Appl Microbiol Biotechnol, 2007. 76(3): p. 561-8).

### Soluble Transhydrogenase (STH)

The FMR enzymes described above, for example, require NADPH as the reduced cofactor. In order to use the PTDH enzyme described elsewhere herein to provide reducing power for these FMR enzymes, it is necessary to transfer reducing power from the cytoplasmic NADH pool to the cytoplasmic NADPH pool in the cell. For example, the enzyme soluble transhydrogenase (STH) from Pseudomonas fluorescens catalyzes freely reversible reduction-oxidation reactions between NADH and NADPH, and this enzyme can be expressed in E. coli (Boonstra 2000, App Env Microbiol, 66: 5161-5166).

In one approach that is compatible with the plasmids (pAB101, pAB 102, and pAB 103) and E. coli strains described above, the STH enzyme is expressed in E. coli by cloning the sth gene from P. fluorescens into single copy on the chromosome. For example, this is accomplished by cloning the sth gene under the control of a constitutive promoter in a non-replicating “suicide” vector, such as a derivative of plasmid R6K. For example, a derivative of R6K can be used that: (1) does not express the gene encoding Pi protein that it required for its replication, (2) contains a DNA sequence identical to a portion of the E. coli chromosome that is a suitable substrate for homologous DNA recombination, and (3) contains a selectable marker such as gentamicin resistance that is compatible with the other selectable markers being used in the bacterial strain. Upon transformation into E. coli, selection for gentamicin-resistance will favor the recovery of cells in which the non-replicating plasmid has integrated into the chromosome via homologous recombination. Those skilled in the art will recognize that many different approaches could be used to express the STH protein in E. coli in a manner that is compatible with the expression of the other genes utilized in the invention.

An alternate approach to providing reducing power for the FMR enzymes is to engineer variant proteins in which the NADPH cofactor-binding pocket is mutated to enhance utilization of NADH by the enzyme. Such a strategy has been successful previously with a NADPH-dependent reductase enzyme (Banta 2002, Prot Eng 15: 131-140).

### Cultivation Conditions to Increase Ethanol Yield

Strain AB304 is cultivated in rich medium supplemented with glucose under anaerobic conditions, and supplemented with antibiotics (Amp, Cam, Spec), inducers (IPTG, propionate, arabinose), and chemical substrates (phosphite or AH2QDS) in varying amounts as necessary. Plasmids pAB101, pAB102, and pAB 103 allow the simultaneous, differential expression of FMR, HPS and PHI, and PTDH due to the compatibility of each functional elements on each plasmid, including their replication origins, selectable antibiotic-resistance genes, and inducible promoters. Formate reductase (FMR) is expressed from the Amp-resistant plasmid pAB 101, and its expression is induced with from 0.001 to 1.0 mM IPTG. The genes for the RuMP pathway, HPS and PHI, are expressed from the Cam-resistant plasmid pAB102, and their expression is induced with 0.2 to 50 mM propionate. Phosphite dehydrogenase (PTDH) is expressed from the Spec-resistant plasmid pAB 103, and its expression is induced with 0.002 to 2% L-arabinose. Phosphite substrate for PTDH is provided at millimolar concentrations for the reduction of NAD+ to NADH. Alternatively, AH2QDS is provided at millimolar concentrations instead of, or in addition to, arabinose and phosphite used for the PTDH-catalyzed reaction.

Anaerobic batch cultures are performed essentially as described (Berrios-Rivera et al., Metab Eng, 2002. 4(3): p. 217-29). Briefly, sealed 15 ml glass vials are prepared containing 14 ml LB broth supplemented with 20 g/L glucose and 1 g/L NaHCO3. The vial is inoculated with 0.1 ml of overnight culture, excess air (3 mL) is removed with a syringe, and the vial is incubated in a rotary shaker at 37° C. for 72 hours. Oxygen remaining in the vial is rapidly consumed by the bacteria to produce anaerobic conditions and induce growth by fermentation. Samples are withdrawn for analysis with a syringe at 24 h intervals.

### Analytical Methods

The engineered strain is characterized by directly measuring the production of ethanol, acetate, and carbon dioxide from glucose, and results are compared to the parent E. coli strain, BW25113. The various concentrations of substrates and products in each fermentation sample are measured as follows.

Ethanol and acetate products are separated by gas chromatography using a HP 5890 GC with a Porapak Q packed column and nitrogen as the carrier gas and detected with a flame ionization detector (FID). Samples are prepared for analysis by centrifuging one milliliter aliquots (10 min at 18000 ref) and filtering the supernatant though a 0.2 micrometer membrane to remove cells and particulate debris. One microliter of each sample is injected on the GC. For acetate measurement, samples are acidified with millimolar concentrations of mM HCL prior to injection. Ethanol and acetate standards are used to calibrate the instrument and construct a standard curve for quantitation of each product. The ration of ethanol to acetate is calculated for each sample.

Glucose substrate in the fermentation broth is measured using an assay kit purchased from Sigma Chemical Co. based on glucose oxidase (GO) enzyme. Glucose standard controls are used to calibrate the assay and construct a standard curve for quantitation. The amount of ethanol produced relative to the amount of glucose consumed by the bacteria is calculated as a ratio. Alternatively, glucose concentrations are measured using the dinitrosalicyclic acid (DNS) assay for reducing sugars (Miller, Anal. Chem., 1959. 31(3) p. 426-8). Total cell biomass is determined from dried cell pellets from normalized culture volumes.

Carbon dioxide production is measured by analysis of the culture headspace by gas chromatography using a HP 5890 GC with a Porapak Q column using hydrogen as the carrier gas (30 milliliters per minute) and a thermal conductivity detector (TCD). Samples are prepared by addition of HCL to decrease the pH to 3 and incubation in a 37 C water bath to release any dissolved carbon dioxide into the vapor phase. Standard controls prepared with sodium bicarbonate are used to calibrate the instrument and construct a standard curve for quantitation. Carbon dioxide may also be measured directly via mass spectroscopy (GC/MS). Carbon dioxide may also be measured indirectly and qualitatively by determining the volume of fermentation gasses captured in inverted test tubes in the liquid culture media.

Relative to the parent strain B W15223, cultivation of strain AB304 under the above conditions is found to: (1) produce higher molar ratios of ethanol to acetate than the parent strain BW25113 due to the additional reducing power provided to the cell via oxidation of phosphite or AH2QDS, (2) produce higher molar ratios of ethanol to carbon dioxide due to the reduced production of carbon dioxide from formate, (3) produce higher molar ratios of ethanol relative to consumed glucose due to the conversion of formate into ethanol by the FMR and RuMP pathway enzymes, and/or (4) show and increase in biomass production due to the additional pyruvate and ATP produced from the RuMP pathway.

### Example 5

### Increased Ethanol Yield from E. coli through Enzymatic Lignin Oxidation

When an engineered microorganism containing the FMR and RuMP genes described above is cultivated on a cellulosic feedstock, reducing power for production of ethanol from formate can be obtained through enzymatic oxidation of the lignin contained in the feedstock.

Several unique oxidoreductase enzymes have been discovered that catalyze lignin oxidation coupled with reduction of NAD+, including LigD from Sphingomonas paucimobils SYK-6 (Masai et al., Biosci Biotechnol Biochem, 2007. 71(1): p. 1-15; Sato et al., Appl Environ Microbiol, 2009. 75(16): p. 5195-5201), and several enzymes from Pseudomonas species: GGE-DH1 and GGE-DH2 (Pelmont et al., 1985, Biochimie 67:973-986; Pelmont et al. 1989 FEMS Microbiol Lett 57:109-114), DH (Vicuna et al., Appl Environ Microbiol, 1987. 53(11): p. 2605-2609), and DH-I and DH-II (Habu et al., Agric Biol Chem, 1988. 52(12): p. 3073-3079).

Other microorganisms that catabolize lignin are predicted to produce additional related oxidoreductase enzymes. Such enzymes can be identified for use in the current invention through (1) established experimental approaches for cloning and screening new enzymes for lignin oxidation coupled to NAD+ reduction to NADH, or (2) bioinformatic methods that can predict enzymes that may oxidize lignin coupled with reduction of NAD+ to NADH based on their amino-acid sequence identity to enzymes known to posses this activity and substrate specificity.

DNA encoding lignin-oxidizing enzymes are synthesized from published sequences and cloned downstream of an arabinose-inducible promoter on a multi-copy plasmid, analogous to the construct for PTDH expression discussed elsewhere herein. Expression is induced with arabinose, and cell-free extracts produced by sonication. Enzyme assays are performed as described (Pelmont et al. 1989 FEMS Microbiol Lett 57:109-114) using commercially available model lignin substrates (e.g. guaiacylglycerol-b-guaiacyl ether (GGE) or 4-hydroxy-3-methoxybenzaldehyde (vanillan)). Specific activity is determined from the change in absorbance at 340 nm due to NADH production and protein mass from Bradford assays.

### Example 6

### Electrochemical Bioreactor

Energy to reduce formate into ethanol can be provided to the cell as electric power by using an electrochemical bioreactor. This system has the advantage of neither requiring additional feedstocks as external reductant, nor depositing oxidation products in the spent fermentation broth.

Addition of an electron transport mediator or electron shuttle to the electrochemical bioreactor is anticipated to be required to facilitate a high rate of electron transport between the cathode and the ethanologen. In the case of cellulosic ethanol, lignin fragments may be a convenient choice of electron shuttle, since they are already present in the pretreated feedstock and thus would add little or no cost.

In one embodiment of the invention, the energy contained in the waste streams produced during fermentation of a cellulosic feedstock (Aden et al. 2002. Lignocellulosic Biomass to Ethanol Process Design and Economics Utilizing Co-Current Dilute Acid Prehydrolysis and Enzymatic Hydrolysis for Corn Stover. National Renewable Energy Laboratory, Golden Colo.) is captured through combustion and a boiler/turbogenerator is used to produce electrical energy that is used to drive the production of additional ethanol by the engineered microorganism. Initial calculations suggest that the combustion of fermentation solids gives enough reducing power to increase the ethanol yield by about 12.5% with no additional cost. The full theoretical yield increase of 50% could be achieved by buying additional electricity or increasing the efficiency of power generation, and the additional cost would depend upon the cost per kWh of that electricity.

### Electrochemical Bioreactor Operation.

A “three-compartmented electrochemical bioreactor” (3-CEB) design, as described by Hwang et al. (Hwang et al., Biotechnol Bioprocess Eng, 2008. 13:677-682), is employed which features a cathode made of graphite felt modified with neutral red to transfer electrons directly to cells suspended in the catholyte (FIG. 7). It has the advantages over older 2-CEB designs of keeping evolved O2 away from the cathode chamber, ease of sterilization, and making control of volumes easier. The chamber is operated as described (Hwang et al., Biotechnol Bioprocess Eng, 2008. 13:677-682). Essentially, E. coli growth media is added to the catholyte compartment containing 200 mM glucose as substrate, and an anolyte solution containing 200 mM phosphate buffer at pH 4 and 200 mM NaCl is added to the anolyte compartment. The medium is also supplemented with the following amounts and combinations of redox-active compounds that can serve as electron mediators: 10 uM AQDS, 10 uM AQDS and 5% lignin sulfate, 5% lignin sulfate, 10 uM FeSO4, 10 uM FeSO4 and 5% lignin sulfate. The catholyte compartment is inoculated with freshly prepared, washed, early stationary phase cells of strain AB304 containing plasmids pAB101 and pAB102 at 10E9 CFU per ml. The catholyte compartment is also supplemented with antibiotics (Amp, Cam) and inducers (IPTG, propionate) in varying amounts as necessary to induce expression of FMR and the RuMP enzymes (HPS and PHI), respectively. The reactor is incubated at 37° C. under anaerobic or microaerobic conditions with and without an applied voltage of 1.5V to 3.0V DC, which is sufficient to supply reducing equivalents to the cellular NADH system (Hwang et al., Biotechnol Bioprocess Eng, 2008. 13:677-682). Samples are continuously removed from the “outlet compartment”, containing cells and metabolites (ethanol, acetate and CO₂), which pass through the porous glass separating the outlet chamber from the cathodic chamber. Samples are analyzed using the methods described elsewhere herein.

Enhanced ethanol production (above that produced by the BW25113 control strain under similar conditions) is found to be stimulated by, and proportional to, the concentration of reduced electron mediator added to the reactor. When the concentration of added mediator is very low (<1 mM), significant ethanol production is only detected when a voltage is applied to the bioreactor to continuously regenerate the reduced form of the mediator.

### Example 7

### System Optimization

Realized benefits (CO₂ reduction and increased ethanol yield) depend on the extent and efficiency with which reducing power is provided to the cell, and the optimization of carbon flux through the engineered pathways. Therefore, the invention includes methods and compositions to (1) create enhanced enzymes with improved properties, and (2) optimize both native and engineered metabolic pathways and carbon fluxes to ethanol. The results from these experiments can serve as a guide to further work for optimizing the organism and enzymes, and evaluating process scalability.

By way of a non-limiting example of expression and optimization of lignin oxidizing enzymes, E. coli can be used to leverage the extensive knowledge and tools for genetic manipulation and protein expression in this organism. Purified enzyme (expressed by secretion) can be assayed on partially degraded lignin to determine the magnitude of enzyme improvement necessary for success. Improvements can be achieved by multiple parallel strategies including increasing expression and optimizing activity through “directed evolution” and rational design. Performance goals include, but are not limited to, increased specific activity, enhanced substrate specificity, improved expression, and improved stability.

By way of a non-limiting example of metabolic engineering and optimization, genes encoding the RuMP pathway can be cloned and expressed, using expression of individual enzymes to confirm enzyme activity. Carbon fluxes through the RuMP pathway can be measured with radioactive tracers to validate pathway function, identify undesirable branches and bottlenecks, and guide pathway optimization. Pathway optimization may also be performed using mutagenesis and fitness screening via muliplexed fluorescence activated cell sorting (FACS).

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of reducing production of CO₂ in a fermentation process of producing an alcohol, said method comprising incubating a microorganism in a culture medium, wherein said culture medium comprises fermentable and non-fermentable portions, and further wherein the non-fermentable portion of said culture medium can be oxidized by the microorganism thereby minimizing the need for oxidation of the fermentable portion.

2. The method of claim 1, wherein said alcohol is ethanol or butanol.

3. The method of claim 1, wherein the yield of ethanol production is increased.

4. The method of claim 1, wherein the non-fermentable portion comprises lignin.

5. The method of claim 1, wherein the fermentable portion comprises carbohydrates.

6. The method of claim 1, wherein said microorganism has been modified to eliminate production of CO₂ from formate.

7. The method of claim 6, wherein said modification is the inactivation of formate-hydrogen lyase (FHL) and formate dehydrogenase (FDH).

8. The method of claim 6, wherein the microorganism is further modified to express a component of a pathway that converts formate to formaldehyde.

9. The method of claim 8, wherein said microorganism has been modified to express formate reductase (FMR).

10. The method of claim 8, wherein said microorganism has been further modified to assimilate carbon from a one-carbon compound.

11. The method of claim 10, wherein said modification comprises expressing a component of the ribulose monophosphate pathway.

12. The method of claim 11, wherein said component of the ribulose monophosphate pathway is hexulose phosphate synthase (HPS) and phosphohexulose isomerase (PHI).

13. The method of claim 10, wherein said microorganism has been further modified to express an oxidoreductase enzyme wherein said enzyme catalyzes the oxidation of said non-fermentable portion of the culture medium to support the conversion of oxidized biological cofactors to reduced cofactors.

14. The method of claim 13, wherein said enzyme is able to oxidize lignin.

15. The method of claim 13, wherein said enzyme is phosphate dehydrogenase (PTDH).

16. The method of claim 1, wherein said microorganism is cultured in an electrochemical bioreactor.

17. The method of claim 16, wherein said microorganism is modified to produce an electron shuttle that is secreted outside the microorgansim, and wherein said electron shuttle is capable of transferring electrons to the cell to support the intracellular conversion of oxidized biological cofactors to reduced cofactors.

18. The method of claim 17, wherein said electron shuttle is a small molecule.

19. The method of claim 17, wherein said electron shuttle is a protein.

20. The method of claim 1, wherein said microorganism has been modified to reduce or eliminate production of carbon dioxide from pyruvate by inactivating pyruvate decarboxylase (PDC).

21. The method of claim 1, wherein said microorganism has been modified to reduce or eliminate production of carbon dioxide from pyruvate by inactivating pyruvate-ferredoxin oxidoreductase (PFO).

22. The method of claim 1, wherein said microorganism has been modified to reduce or eliminate production of carbon dioxide from pyruvate by inactivating pyruvate dehydrogenase.

23. The method of claim 1, wherein said microorganism has been modified to reduce or eliminate production of any one or more of carbon dioxide from pyruvate by inactivating pyruvate decarboxylase (PDC), carbon dioxide from pyruvate by inactivating pyruvate-ferredoxin oxidoreductase (PFO), or carbon dioxide from pyruvate by inactivating pyruvate dehydrogenase, further wherein the microorganism has been modified to enable conversion of pyruvate to acetyl-CoA for production of formate instead of carbon dioxide.

24. The method of claim 23, wherein said modification to enable conversion of pyruvate to acetyl-CoA comprises expression of pyruvate-formate lyase (PFL).

25. The method of claim 1, wherein the microorganism is modified to prevent production of carbon dioxide from formate by inactivating formate dehydrogenase (FDH).

26. The method of claim 25, wherein the microorganism is further modified to express an enzyme that converts formate to formaldehyde.

27. The method of claim 26, wherein said enzyme is formate reductase.

28. The method of claim 1, wherein said microorganism has been modified to utilize the ribulose monophosphate pathway to convert three formaldehyde molecules into glyceraldehyde-3-phosphate.

29. The method of claim 1, wherein said microorganism has been modified to utilize the serine pathway to assimilate carbon from formaldehyde and carbon dioxide into 3-phosphoglycerate.

30. The method of claim 29, wherein said microorganism has been further modified to express an oxidoreductase enzyme wherein said enzyme catalyzes the oxidation of said non-fermentable portion of the culture medium to support the conversion of oxidized biological cofactors to reduced cofactors.

31. The method of claim 30, wherein said enzyme is able to oxidize lignin.

32. The method of claim 31, wherein said enzyme is phosphate dehydrogenase (PTDH).

33. The method of claim 1, wherein said microorganism has been modified to utilize the Calvin cycle to convert six carbon dioxide molecules into fructose-6-phosphate.

34. The method of claim 4, wherein said lignin is modified in either a chemical or biological process to be more oxidizable by the microorganism.

35. A microorganism modified to permit the reduced production of CO₂ in a fermentation process, wherein said modification is the activation of an oxidoreductase enzyme, wherein said enzyme is capable of catalyzing the oxidation of lignin.
